SEQUENCE LISTING (66 sequences)

SEQ ID NO: 1 - DNA, 1560 bp, Veillonella dispar
ttggagagtttgatcctggctcaggacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaacctcggcatcgaggatggatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaatagttagaaggattgacggtaccggaatagaaagccacggctaactacgtgccagcagccgcggtaatacgtaggtggcaagcgttgtccggaattattgggcgtaaagcgcgcgcaggcggattggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaactagagatagttcctcttcttcggaagccagaaaacaggtggtgcacggttgtcgtcagctcgtgtcgtgagatgttgggttaagtcccgcaacgagcgcaacccctatcttatgttgccagcacttcgggtgggaactcatgagagactgccgcagacaatgcggaggaaggcggggatgacgtcaaatcatcatgccccttatgacctgggctacacacgtactacaatgggagttaatagacggaagcaataccgcgaggtggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagccgtatcggaaggtgcggctggatcacctccttt

SEQ ID NO: 2 - DNA, 32 bp, Veillonella dispar
aagagcgatggaagcttgcttctatcaatctt

SEQ ID NO: 3 - DNA, 80 bp, Veillonella dispar
acgatctaacctcggcatcgaggatggatgaaaggtggcctctatttataagctatcactgaaggaggggattgcgtctg

SEQ ID NO: 4 - DNA, 64 bp, Veillonella dispar
gccttcgggttgtaaagctctgttaatcgggacgaaaggccttcttgcgaatagttagaaggat

SEQ ID NO: 5 - DNA, 84 bp, Veillonella dispar
attggtcagtctgtcttaaaagttcggggcttaaccccgtgatgggatggaaactgccaatctagagtatcggagaggaaagtg

SEQ ID NO: 6 - DNA, 43 bp, Veillonella dispar
ggtactaggtgtaggaggtatcgaccccttctgtgccggagtt

SEQ ID NO: 7 - DNA, 51 bp, Veillonella dispar
cattgatggacagaactagagatagttcctcttcttcggaagccagaaaac

SEQ ID NO: 8 - DNA, 60 bp, Veillonella dispar
ctatcttatgttgccagcacttcgggtgggaactcatgagagactgccgcagacaatgcg

SEQ ID NO: 9 - DNA, 58 bp, Veillonella dispar
ggagttaatagacggaagcaataccgcgaggtggagcaaacccgagaaacactctctc

SEQ ID NO: 10 - DNA, 45 bp, Veillonella dispar
caccacgaaagtcggaagtgcccaaagccggtggggtaaccttcg

SEQ ID NO: 11 - DNA, 1560 bp, Veillonella parvula
ttggagagtttgatcctggctcaggacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaatctcggcatcgaggaaagatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaacagttagaaggattgacggtaccggaatagaaagccacggctaactacgtgccagcagccgcggtaatacgtaggtggcaagcgttgtccggaattattgggcgtaaagcgcgcgcaggcggatcagtcagtctgtcttaaaagttcggggcttaaccccgtgatgggatggaaactgctgatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaaccagagatggttcctcttcttcggaagccagaaaacaggtggtgcacggttgtcgtcagctcgtgtcgtgagatgttgggttaagtcccgcaacgagcgcaacccctatcttatgttgccagcactttgggtggggactcatgagagactgccgcagacaatgcggaggaaggcggggatgacgtcaaatcatcatgccccttatgacctgggctacacacgtactacaatgggagttaatagacggaagcgagatcgcgagatggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagccgtatcggaaggtgcggctggatcacctccttt

SEQ ID NO: 12 - DNA, 32 bp, Veillonella parvula; identical to SEQ ID NO: 2

SEQ ID NO: 13 - DNA, 80 bp, Veillonella parvula
acgatctaatctcggcatcgaggaaagatgaaaggtggcctctatttataagctatcactgaaggaggggattgcgtctg

SEQ ID NO: 14 - DNA, 64 bp, Veillonella parvula
gccttcgggttgtaaagctctgttaatcgggacgaaaggccttcttgcgaacagttagaaggat

SEQ ID NO: 15 - DNA, 84 bp, Veillonella parvula
atcagtcagtctgtcttaaaagttcggggcttaaccccgtgatgggatggaaactgctgatctagagtatcggagaggaaagtg

SEQ ID NO: 16 - DNA, 43 bp, Veillonella parvula; identical to SEQ ID NO: 6

SEQ ID NO: 17 - DNA, 51 bp, Veillonella parvula
cattgatggacagaaccagagatggttcctcttcttcggaagccagaaaac

SEQ ID NO: 18 - DNA, 60 bp, Veillonella parvula
ctatcttatgttgccagcactttgggtggggactcatgagagactgccgcagacaatgcg

SEQ ID NO: 19 - DNA, 58 bp, Veillonella parvula
ggagttaatagacggaagcgagatcgcgagatggagcaaacccgagaaacactctctc

SEQ ID NO: 20 - DNA, 45 bp, Veillonella parvula; identical to SEQ ID NO: 10

SEQ ID NO: 21 - DNA, 1560 bp, Veillonella atypica
ttggagagtttgatcctggctcaggacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatccaatctcggcatcgagactggatgaaaggtggcctctatttataagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaatggttcttgtgcgaatagtgcgaggatttgacggtaccggaatagaaagccacggctaactacgtgccagcagccgcggtaatacgtaggtggcaagcgttgtccggaattattgggcgtaaagcgcgcgcaggcggattggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaactagagatagttcctcttcttcggaagccagaaaacaggtggtgcacggttgtcgtcagctcgtgtcgtgagatgttgggttaagtcccgcaacgagcgcaacccctatcttatgttgccagcacttcgggtgggaactcatgagagactgccgcagacaatgcggaggaaggcggggatgacgtcaaatcatcatgccccttatgacctgggctacacacgtactacaatgggagttaatagacggaagcgaaaccgcgaggtggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagccgtatcggaaggtgcggctggatcacctccttt

SEQ ID NO: 22 - DNA, 32 bp, Veillonella atypica; identical to SEQ ID NO: 2

SEQ ID NO: 23 - DNA, 80 bp, Veillonella atypica
acgatccaatctcggcatcgagactggatgaaaggtggcctctatttataagctatcactgaaggaggggattgcgtctg

SEQ ID NO: 24 - DNA, 64 bp, Veillonella atypica
gccttcgggttgtaaagctctgttaatcgggacgaatggttcttgtgcgaatagtgcgaggatt

SEQ ID NO: 25 - DNA, 84 bp, Veillonella atypica; identical to SEQ ID NO: 5

SEQ ID NO: 26 - DNA, 43 bp, Veillonella atypica; identical to SEQ ID NO: 6

SEQ ID NO: 27 - DNA, 51 bp, Veillonella atypica; identical to SEQ ID NO: 7

SEQ ID NO: 28 - DNA, 60 bp, Veillonella atypica; identical to SEQ ID NO: 8

SEQ ID NO: 29 - DNA, 58 bp, Veillonella atypica
ggagttaatagacggaagcgaaaccgcgaggtggagcaaacccgagaaacactctctc

SEQ ID NO: 30 - DNA, 45 bp, Veillonella atypica; identical to SEQ ID NO: 10

SEQ ID NO: 31 - DNA, 65 bp, Veillonella dispar
ttggagagtttgatcctggctcaggacgaacgctggcggcgtgcttaacacatgcaagtcgaacg

SEQ ID NO: 32 - DNA, 81 bp, Veillonella dispar
agtggcgaacgggtgagtaacgcgtaatcaacctgcccttcagagggggacaacagttggaaacgactgctaataccgcat

SEQ ID NO: 33 - DNA, 174 bp, Veillonella dispar
attagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacg

SEQ ID NO: 34 - DNA, 109 bp, Veillonella dispar
tgacggtaccggaatagaaagccacggctaactacgtgccagcagccgcggtaatacgtaggtggcaagcgttgtccggaattattgggcgtaaagcgcgcgcaggcgg

SEQ ID NO: 35 - DNA, 148 bp, Veillonella dispar
gaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatg

SEQ ID NO: 36 - DNA, 131 bp, Veillonella dispar
aacgcaataagtaccccgcctggggagtacgaccgcaaggttgaaactcaaaggaattgacgggggcccgcacaagcggtggagtatgtggtttaattcgacgcaacgcgaagaaccttaccaggtcttga

SEQ ID NO: 37 - DNA, 69 bp, Veillonella dispar
aggtggtgcacggttgtcgtcagctcgtgtcgtgagatgttgggttaagtcccgcaacgagcgcaaccc

SEQ ID NO: 38 - DNA, 67 bp, Veillonella dispar
gaggaaggcggggatgacgtcaaatcatcatgccccttatgacctgggctacacacgtactacaatg

SEQ ID NO: 39 - DNA, 110 bp, Veillonella dispar
agttcggatcgtaggctgcaactcgcctacgtgaagtcggaatcgctagtaatcgcaggtcagcatactgcggtgaatacgttcccgggccttgtacacaccgcccgtca

SEQ ID NO: 40 - DNA, 89 bp, Veillonella dispar
ggagccagccgtctaaggtaaagtcgatgattggggtgaagtcgtaacaaggtagccgtatcggaaggtgcggctggatcacctccttt

SEQ ID NO: 41 - DNA, 65 bp, Veillonella parvula; identical to SEQ ID NO: 31

SEQ ID NO: 42 - DNA, 81 bp, Veillonella parvula; identical to SEQ ID NO: 32

SEQ ID NO: 43 - DNA, 174 bp, Veillonella parvula; identical to SEQ ID NO: 33

SEQ ID NO: 44 - DNA, 109 bp, Veillonella parvula; identical to SEQ ID NO: 34

SEQ ID NO: 45 - DNA, 148 bp, Veillonella parvula; identical to SEQ ID NO: 35

SEQ ID NO: 46 - DNA, 131 bp, Veillonella parvula; identical to SEQ ID NO: 36

SEQ ID NO: 47 - DNA, 69 bp, Veillonella parvula; identical to SEQ ID NO: 37

SEQ ID NO: 48 - DNA, 67 bp, Veillonella parvula; identical to SEQ ID NO: 38

SEQ ID NO: 49 - DNA, 110 bp, Veillonella parvula; identical to SEQ ID NO: 39

SEQ ID NO: 50 - DNA, 89 bp, Veillonella parvula; identical to SEQ ID NO: 40

SEQ ID NO: 51 - DNA, 65 bp, Veillonella atypica; identical to SEQ ID NO: 31

SEQ ID NO: 52 - DNA, 81 bp, Veillonella atypica; identical to SEQ ID NO: 32

SEQ ID NO: 53 - DNA, 174 bp, Veillonella atypica; identical to SEQ ID NO: 33

SEQ ID NO: 54 - DNA, 109 bp, Veillonella atypica; identical to SEQ ID NO: 34

SEQ ID NO: 55 - DNA, 148 bp, Veillonella atypica; identical to SEQ ID NO: 35

SEQ ID NO: 56 - DNA, 131 bp, Veillonella atypica; identical to SEQ ID NO: 36

SEQ ID NO: 57 - DNA, 69 bp, Veillonella atypica; identical to SEQ ID NO: 37

SEQ ID NO: 58 - DNA, 67 bp, Veillonella atypica; identical to SEQ ID NO: 38

SEQ ID NO: 59 - DNA, 110 bp, Veillonella atypica; identical to SEQ ID NO: 39

SEQ ID NO: 60 - DNA, 89 bp, Veillonella atypica; identical to SEQ ID NO: 40

SEQ ID NO: 61 - DNA, 1571 bp, Lactobacillus plantarum
attaatttgagagtttgatcctggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtaggagaacctgcggctggatcacctcctttct

SEQ ID NO: 62 - DNA, 1559 bp, Lactobacillus acidophilus
cgagagtttgatcctggctcaggacgaacgctggcggcgtgcctaatacatgcaagtcgagcgagctgaaccaacagattcacttcggtgatgacgttgggaacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtagggtaacggcctaccaaggcaatgatgcatagccgagttgagagactgatcggccacattgggactgagacacggcccaaactcctacgggaggcagcagtagggaatcttccacaatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccgaggaactgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagtgcaatccgtagagatacggagttcccttcggggacactaagacaggtggtgcatggctgtcgtcagctcgtgtcgtgagatgttgggttaagtcccgcaacgagcgcaacccttgtcattagttgccagcattaagttgggcactctaatgagactgccggtgacaaaccggaggaaggtggggatgacgtcaagtcatcatgccccttatgacctgggctacacacgtgctacaatggacagtacaacgaggagcaagcctgcgaaggcaagcgaatctcttaaagctgttctcagttcggactgcagtctgcaactcgactgcacgaagctggaatcgctagtaatcgcggatcagcacgccgcggtgaatacgttcccgggccttgtacacaccgcccgtcacaccatgggagtctgcaatgcccaaagccggtggcctaaccttcgggaaggagccgtctaaggcagggcagatgactggggtgaagtcgtaacaaggtagccgtaggagaacctgcggctggatcacctccttt

SEQ ID NO: 63 - DNA, 1566 bp, Lactobacillus rhamnosus
tatgagagtttgatcctggctcaggatgaacgctggcggcgtgcctaatacatgcaagtcgaacgagttctgattattgaaaggtgcttgcatcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacctgcggctggatcacctcctt

SEQ ID NO: 64 - DNA, 1523 bp, Bifidobacterium longum
gtggagggttcgattctggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacctccttt

SEQ ID NO: 65 - DNA, 1535 bp, Bifidobacterium lactis
gtggagggttcgattctggctcaggatgaacgctggcggcgtgcttaacacatgcaagtcgaacgggatccctggcagcttgctgtcggggtgagagtggcgaacgggtgagtaatgcgtgaccaacctgccctgtgcaccggaatagctcctggaaacgggtggtaataccggatgctccgctccatcgcatggtggggtgggaaatgcttttgcggcatgggatggggtcgcgtcctatcagcttgttggcggggtgatggcccaccaaggcgttgacgggtagccggcctgagagggtgaccggccacattgggactgagatacggcccagactcctacgggaggcagcagtggggaatattgcacaatgggcgcaagcctgatgcagcgacgccgcgtgcgggatggaggccttcgggttgtaaaccgcttttgttcaagggcaaggcacggtttcggccgtgttgagtggattgttcgaataagcaccggctaactacgtgccagcagccgcggtaatacgtagggtgcgagcgttatccggatttattgggcgtaaagggctcgtaggcggttcgtcgcgtccggtgtgaaagtccatcgcctaacggtggatctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggccctttccacgggtcccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgtgccggatcgccgtggagacacggtttcccttcggggccggttcacaggtggtgcatggtcgtcgtcagctcgtgtcgtgagatgttgggttaagtcccgcaacgagcgcaaccctcgccgcatgttgccagcgggtgatgccgggaactcatgtgggaccgccggggtcaactcggaggaaggtggggatgacgtcagatcatcatgccccttacgtccagggcttcacgcatgctacaatggccggtacaacgcggtgcgacacggtgacgtggggcggatcgctgaaaaccggtctcagttcggatcgcagtctgcaactcgactgcgtgaaggcggagtcgctagtaatcgcggatcagcaacgccgcggtgaatgcgttcccgggccttgtacacaccgcccgtcaagtcatgaaagtgggtagcacccgaagccggtggcccgacccttgtggggggagccgtctaaggtgagactcgtgattgggactaagtcgtaacaaggtagccgtaccggaaggtgcggctggatcacctccttt

SEQ ID NO: 66 - DNA, 1566 bp, Lactobacillus paracasei
tatgagagtttgatcctggctcaggatgaacgctggcggcgtgcctaatacatgcaagtcgaacgagttctcgttgatgatcggtgcttgcaccgagattcaacatggaacgagtggcggacgggtgagtaacacgtgggtaacctgcccttaagtgggggataacatttggaaacagatgctaataccgcatagatccaagaaccgcatggttcttggctgaaagatggcgtaagctatcgcttttggatggacccgcggcgtattagctagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacctgcggctggatcacctcctt

(19) United States
(12) Patent Application Publication
(10) Pub. No.: US 2022/0403324 A1
(43) Pub. Date: Dec. 22, 2022

(54) COMPOSITIONS FOR IMPROVING ATHLETIC PERFORMANCE AND METHODS OF USE THEREOF

(71) Applicant: FITBIOMICS INC., New York, NY (US)

(72) Inventor: Jonathan SCHEIMAN, New York, NY (US)

(21) Appl. No.: 17/779,676

(22) PCT Filed: Nov. 25, 2020
(86) PCT No.: PCT/US2020/062336

§ 371 Date: May 25, 2022

Related U.S. Application Data
(60) Provisional application No. 63/018,697, filed on May 1, 2020.
(60) Provisional application No. 62/989,226, filed on Mar. 13, 2020.
(60) Provisional application No. 62/939,793, filed on Nov. 25, 2019.

(51) Int. Cl. C12N 1/20; A61K 35/747; A61P 21/00

(52) U.S. Cl. CPC C12N 1/205; A61K 35/747; A61P 21/00; C12R 2001/25

(57) ABSTRACT

The disclosure provides novel microbial strains, such as Veillonella sp. strains, and compositions comprising the strains that are capable of converting lactate to propionate and acetate. The disclosure also provides compositions comprising the Veillonella sp. strains and lactate producing bacteria, such as Lactobacillus sp. strains and Bifidobacterium sp. strains. The disclosure further provides methods of improving athletic performance, enhancing exercise endurance and reducing inflammation in a subject upon administration of the disclosed strains or compositions.

### CROSS REFERENCE TO RELATED APPLICATIONS

This application claims the benefit of the U.S. Provisional Patent Application Ser. No. 62/939,793 filed Nov. 25, 2019, the U.S. Provisional Patent Application Ser. No. 62/989,226 filed Mar. 13, 2020, and the U.S. Provisional Patent Application Ser. No. 63/018,697 filed May 1, 2020, the contents of each of which is herein incorporated by reference in its entirety for all purposes.

### FIELD

The present disclosure relates to compositions comprising one or more microbial strains, and methods of use thereof in improving athletic performance.

### INCORPORATION OF SEQUENCE LISTING

The contents of the text file submitted electronically herewith are incorporated by reference in their entirety: a computer readable format copy of the Sequence Listing (filename: “FIBI_001_01WO_SeqList_ST25”, date recorded: Nov. 25, 2020, file size: 31.3 kilobytes).

### BACKGROUND

Human gastrointestinal microbiota, also known as gut flora or gut microbiota, comprise microorganisms that live in the digestive tracts of humans, and play a critical role in human health. These microorganisms serve a wide range of functions, such as, defending against pathogens, fortifying host defense by developing and maintaining the intestinal epithelium, inducing antibody production, metabolizing indigestible compounds in food, and training the developing immune system. The gut microbiome also plays a role in the biochemical signaling between the gastrointestinal tract and the central nervous system, referred to as the gut-brain axis.

Formulations comprising microorganisms are used as dietary supplements, and are deemed to provide health benefits by restoring or improving the gut microflora. These microbes may be either resident or transient; resident microbial strains live and reproduce in the digestive tract, while transient microbial strains are introduced into the body through ingested food or by means of dietary supplements. Formulations comprising microorganisms are thought to have immense potential in improving human health.

Formulations comprising microorganisms may have unexplored potential in improving other human functions, such as endurance and athleticism. Pharmaceuticals that improve endurance and athleticism are in popular use. However, there remains a need to develop compositions comprising microorganisms that helps improve endurance and athleticism, for instance, promote or accelerate athletic training, performance and recovery.

### SUMMARY

The disclosure provides isolated and purified strains of Veillonella dispar, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 98.6% sequence identity to SEQ ID NO: 2; (ii) VR2 comprises a nucleic acid sequence with at least 98.6% sequence identity to SEQ ID NO: 3; (iii) VR3 comprises a nucleic acid sequence with at least 98.6% sequence identity to SEQ ID NO: 4; (iv) VR4 comprises a nucleic acid sequence with at least 98.6% sequence identity to SEQ ID NO: 5; (v) VR5 comprises a nucleic acid sequence with at least 98.6% sequence identity to SEQ ID NO: 6; (vi) VR6 comprises a nucleic acid sequence with at least 98.6% sequence identity to SEQ ID NO: 7; (vii) VR7 comprises a nucleic acid sequence with at least 98.6% sequence identity to SEQ ID NO: 8; (viii) VR8 comprises a nucleic acid sequence with at least 98.6% sequence identity to SEQ ID NO: 9; and (ix) VR9 comprises a nucleic acid sequence with at least 98.6% sequence identity to SEQ ID NO: 10.

In some embodiments, the nucleic acid sequence comprises at least one constant region (CR) selected from the group consisting of: CR1, CR2, CR3, CR4, CR5, CR6, CR7, CR8, CR9, CR10, and any combination thereof, wherein (i) CR1 comprises a nucleic acid sequence with at least 80% sequence identity to SEQ ID NO: 31; (ii) CR2 comprises a nucleic acid sequence with at least 80% sequence identity to SEQ ID NO: 32; (iii) CR3 comprises a nucleic acid sequence with at least 80% sequence identity to SEQ ID NO: 33; (iv) CR4 comprises a nucleic acid sequence with at least 80% sequence identity to SEQ ID NO:34; (v) CR5 comprises a nucleic acid sequence with at least 80% sequence identity to SEQ ID NO: 35; (vi) CR6 comprises a nucleic acid sequence with at least 80% sequence identity to SEQ ID NO: 36; (vii) CR7 comprises a nucleic acid sequence with at least 80% sequence identity to SEQ ID NO: 37; (viii) CR8 comprises a nucleic acid sequence with at least 80% sequence identity to SEQ ID NO: 38; (ix) CR9 comprises a nucleic acid sequence with at least 80% sequence identity to SEQ ID NO: 39; and (x) CR10 comprises a nucleic acid sequence with at least 80% sequence identity to SEQ ID NO: 40.

In some embodiments, the nucleic acid sequence comprises at least 98.6% sequence identity to SEQ ID NO: 1. The disclosure also provides isolated and purified strains of Veillonella dispar, comprising: a 16S rRNA gene comprising a nucleic acid sequence with at least 98.6% sequence identity to SEQ ID NO: 1.

The disclosure provides isolated and purified strains of Veillonella parvula, comprising: a 16S rRNA gene comprising a nucleic acid sequence comprising at least one variable region (VR) selected from the group consisting of VR1, VR2, VR3, VR4, VR5, VR6, VR7, VR8, VR9, and any combination thereof, wherein (i) VR1 comprises a nucleic acid sequence with at least 98.6% sequence identity to SEQ ID NO: 12; (ii) VR2 comprises a nucleic acid sequence with at least 98.6% sequence identity to SEQ ID NO: 13; (iii) VR3 comprises a nucleic acid sequence with at least 98.6% sequence identity to SEQ ID NO: 14; (iv) VR4 comprises a nucleic acid sequence with at least 98.6% sequence identity to SEQ ID NO: 15; (v) VR5 comprises a nucleic acid sequence with at least 98.6% sequence identity to SEQ ID NO: 16; (vi) VR6 comprises a nucleic acid sequence with at least 98.6% sequence identity to SEQ ID NO: 17; (vii) VR7 comprises a nucleic acid sequence with at least 98.6% sequence identity to SEQ ID NO: 18; (viii) VR8 comprises a nucleic acid sequence with at least 98.6% sequence identity to SEQ ID NO: 19; and (ix) VR9 comprises a nucleic acid sequence with at least 98.6% sequence identity to SEQ ID NO: 20.

In some embodiments, the nucleic acid sequence comprises at least one constant region (CR) selected from the group consisting of: CR1, CR2, CR3, CR4, CR5, CR6, CR7, CR8, CR9, CR10, and any combination thereof, wherein (i) CR1 comprises a nucleic acid sequence with at least 80% sequence identity to SEQ ID NO: 41; (ii) CR2 comprises a nucleic acid sequence with at least 80% sequence identity to SEQ ID NO: 42; (iii) CR3 comprises a nucleic acid sequence with at least 80% sequence identity to SEQ ID NO: 43; (iv) CR4 comprises a nucleic acid sequence with at least 80% sequence identity to SEQ ID NO: 44; (v) CR5 comprises a nucleic acid sequence with at least 80% sequence identity to SEQ ID NO: 45; (vi) CR6 comprises a nucleic acid sequence with at least 80% sequence identity to SEQ ID NO: 46; (vii) CR7 comprises a nucleic acid sequence with at least 80% sequence identity to SEQ ID NO: 47; (viii) CR8 comprises a nucleic acid sequence with at least 80% sequence identity to SEQ ID NO: 48; (ix) CR9 comprises a nucleic acid sequence with at least 80% sequence identity to SEQ ID NO: 49; and (x) CR10 comprises a nucleic acid sequence with at least 80% sequence identity to SEQ ID NO: 50.

In some embodiments, the nucleic acid sequence comprises at least 98.6% sequence identity to SEQ ID NO: 11. The disclosure also provides isolated and purified strains of Veillonella parvula, comprising: a 16S rRNA gene comprising a nucleic acid sequence with at least 98.6% sequence identity to SEQ ID NO: 11.

The disclosure provides isolated and purified strains of Veillonella atypica, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 98.6% sequence identity to SEQ ID NO: 22; (ii) VR2 comprises a nucleic acid sequence with at least 98.6% sequence identity to SEQ ID NO: 23; (iii) VR3 comprises a nucleic acid sequence with at least 98.6% sequence identity to SEQ ID NO: 24; (iv) VR4 comprises a nucleic acid sequence with at least 98.6% sequence identity to SEQ ID NO: 25; (v) VR5 comprises a nucleic acid sequence with at least 98.6% sequence identity to SEQ ID NO: 26; (vi) VR6 comprises a nucleic acid sequence with at least 98.6% sequence identity to SEQ ID NO: 27; (vii) VR7 comprises a nucleic acid sequence with at least 98.6% sequence identity to SEQ ID NO: 28; (viii) VR8 comprises a nucleic acid sequence with at least 98.6% sequence identity to SEQ ID NO: 29; and (ix) VR9 comprises a nucleic acid sequence with at least 98.6% sequence identity to SEQ ID NO: 30.

In some embodiments, the nucleic acid sequence comprises at least one constant region (CR) selected from the group consisting of CR1, CR2, CR3, CR4, CR5, CR6, CR7, CR8, CR9, CR10 and any combination thereof, wherein (i) CR1 comprises a nucleic acid sequence with at least 80% sequence identity to SEQ ID NO: 51; (ii) CR2 comprises a nucleic acid sequence with at least 80% sequence identity to SEQ ID NO: 52; (iii) CR3 comprises a nucleic acid sequence with at least 80% sequence identity to SEQ ID NO: 53; (iv) CR4 comprises a nucleic acid sequence with at least 80% sequence identity to SEQ ID NO: 54; (v) CR5 comprises a nucleic acid sequence with at least 80% sequence identity to SEQ ID NO: 55; (vi) CR6 comprises a nucleic acid sequence with at least 80% sequence identity to SEQ ID NO: 56; (vii) CR7 comprises a nucleic acid sequence with at least 80% sequence identity to SEQ ID NO: 57; (viii) CR8 comprises a nucleic acid sequence with at least 80% sequence identity to SEQ ID NO: 58; (ix) CR9 comprises a nucleic acid sequence with at least 80% sequence identity to SEQ ID NO: 59; and (x) CR10 comprises a nucleic acid sequence with at least 80% sequence identity to SEQ ID NO: 60.

In some embodiments, the nucleic acid sequence comprises at least 98.6% sequence identity to SEQ ID NO: 21. The disclosure also provides isolated and purified strains of Veillonella atypica, comprising: a 16S rRNA gene comprising a nucleic acid sequence with at least 98.6% sequence identity to SEQ ID NO: 21.

The disclosure further provides an isolated and purified Veillonella dispar having deposit accession number PTA-126861, or a strain having all of the identifying characteristics of Veillonella dispar PTA-126861, or a mutant thereof. The disclosure also provides an isolated and purified Veillonella parvula having deposit accession number PTA-126859, or a strain having all of the identifying characteristics of Veillonella parvula PTA-126859, or a mutant thereof. Further, the disclosure provides an isolated and purified Veillonella atypica having deposit accession number PTA-126860, or a strain having all of the identifying characteristics of Veillonella atypica PTA-126860, or a mutant thereof.

The disclosure provides compositions comprising any one or more of the Veillonella dispar strains disclosed herein. The disclosure provides compositions comprising any one or more of the Veillonella parvula strains disclosed herein. The disclosure provides compositions comprising any one or more of the Veillonella atypica strains disclosed herein.

The disclosure provides compositions comprising any one or more of the Veillonella sp. strains disclosed herein. In some embodiments, the composition comprises one or more lactate producing bacteria. In some embodiments, the lactate-producing bacteria belongs to the genus Lactobacillus or Bifidobacterium. In some embodiments, the lactate-producing bacteria is Lactobacillus plantarum, Lactobacillus acidophilus, Lactobacillus rhamnosus, Lactobacillus paracasei, Bifidobacterium longum, Bifidobacterium lactis, or any combination thereof. In some embodiments, the composition produces more acetate than the Veillonella sp. strain. In some embodiments, the composition is a food composition, a beverage composition or a dietary supplement composition. In some embodiments, the composition comprises a pharmaceutically acceptable carrier.

The disclosure provides compositions comprising any one or more of the Veillonella sp. strains disclosed herein, and any one or more of the following strains: (a) an isolated and purified Lactobacillus plantarum comprising a 16S rRNA nucleic acid sequence that is at least 97% identical to SEQ ID NO: 61; (b) an isolated and purified Lactobacillus acidophilus comprising a 16S rRNA nucleic acid sequence that is at least 97% identical to SEQ ID NO: 62; (c) an isolated and purified Lactobacillus rhamnosus comprising a 16S rRNA nucleic acid sequence that is at least 97% identical to SEQ ID NO: 63; (d) an isolated and purified Bifidobacterium longum comprising a 16S rRNA nucleic acid sequence that is at least 97% identical to SEQ ID NO: 64; (e) an isolated and purified Bifidobacterium lactis comprising a 16S rRNA nucleic acid sequence that is at least 97% identical to SEQ ID NO: 65; and (f) an isolated and purified Lactobacillus paracasei comprising a 16S rRNA nucleic acid sequence that is at least 97% identical to SEQ ID NO: 66.

The disclosure provides compositions comprising any one or more of the Veillonella sp. strains disclosed herein, and any one or more of the following strains: (a) an isolated and purified Lactobacillus plantarum comprising a 16S rRNA nucleic acid sequence that is at least 97% identical to SEQ ID NO: 61; (b) an isolated and purified Lactobacillus acidophilus comprising a 16S rRNA nucleic acid sequence that is at least 97% identical to SEQ ID NO: 62; (c) an isolated and purified Lactobacillus rhamnosus comprising a 16S rRNA nucleic acid sequence that is at least 97% identical to SEQ ID NO: 63; and (d) an isolated and purified Lactobacillus paracasei comprising a 16S rRNA nucleic acid sequence that is at least 97% identical to SEQ ID NO: 66.

The disclosure provides compositions comprising: (a) an isolated and purified Veillonella atypica having deposit accession number PTA-126860, or a strain having all of the identifying characteristics of Veillonella atypica PTA-126860, or a mutant thereof, (b) an isolated and purified Lactobacillus plantarum comprising a 16S rRNA nucleic acid sequence that is at least 97% identical to SEQ ID NO: 61; (c) an isolated and purified Lactobacillus acidophilus comprising a 16S rRNA nucleic acid sequence that is at least 97% identical to SEQ ID NO: 62; (d) an isolated and purified Lactobacillus rhamnosus comprising a 16S rRNA nucleic acid sequence that is at least 97% identical to SEQ ID NO: 63; (e) an isolated and purified Bifidobacterium longum comprising a 16S rRNA nucleic acid sequence that is at least 97% identical to SEQ ID NO: 64; and (f) an isolated and purified Bifidobacterium lactis comprising a 16S rRNA nucleic acid sequence that is at least 97% identical to SEQ ID NO: 65.

The disclosure provides compositions comprising: (a) an isolated and purified Veillonella atypica having deposit accession number PTA-126860, or a strain having all of the identifying characteristics of Veillonella atypica PTA-126860, or a mutant thereof, (b) an isolated and purified Lactobacillus plantarum comprising a 16S rRNA nucleic acid sequence that is at least 97% identical to SEQ ID NO: 61; (c) an isolated and purified Lactobacillus acidophilus comprising a 16S rRNA nucleic acid sequence that is at least 97% identical to SEQ ID NO: 62; and (d) an isolated and purified Lactobacillus rhamnosus comprising a 16S rRNA nucleic acid sequence that is at least 97% identical to SEQ ID NO: 63.

The disclosure provides methods of altering the microbiome of a subject, comprising administering an effective dose of any one or more of the Veillonella dispar strains disclosed herein; any one or more of the Veillonella parvula strains disclosed herein; any one or more of the Veillonella atypica strains disclosed herein; and/or any one or more of the compositions disclosed herein (comprising one or more of the aforementioned bacteria) to the subject.

The method also provides methods of reducing the level of lactic acid and/or lactates in blood of a subject, comprising administering an effective dose of any one or more of the Veillonella dispar strains disclosed herein; any one or more of the Veillonella parvula strains disclosed herein; any one or more of the Veillonella atypica strains disclosed herein; and/or any one or more of the compositions disclosed herein (comprising one or more of the aforementioned bacteria) to the subject. The disclosure provides methods of increasing the level of propionic acid and/or propionates in blood of a subject, comprising administering an effective dose of any one or more of the Veillonella dispar strains disclosed herein; any one or more of the Veillonella parvula strains disclosed herein; any one or more of the Veillonella atypica strains disclosed herein; and/or any one or more of the compositions disclosed herein (comprising one or more of the aforementioned bacteria) to the subject. The disclosure provides methods of increasing the level of acetic acid and/or acetates in blood of a subject, comprising administering an effective dose of any one or more of the Veillonella dispar strains disclosed herein; any one or more of the Veillonella parvula strains disclosed herein; any one or more of the Veillonella atypica strains disclosed herein; and/or any one or more of the compositions disclosed herein (comprising one or more of the aforementioned bacteria) to the subject.

The disclosure provides methods of enhancing exercise endurance of a subject, comprising administering an effective dose of any one or more of the Veillonella dispar strains disclosed herein; any one or more of the Veillonella parvula strains disclosed herein; any one or more of the Veillonella atypica strains disclosed herein; and/or any one or more of the compositions disclosed herein (comprising one or more of the aforementioned bacteria) to the subject. The disclosure provides methods of improving the athletic performance of a subject, comprising administering an effective dose of any one or more of the Veillonella dispar strains disclosed herein; any one or more of the Veillonella parvula strains disclosed herein; any one or more of the Veillonella atypica strains disclosed herein; and/or any one or more of the compositions disclosed herein (comprising one or more of the aforementioned bacteria) to the subject.

The disclosure provides methods of reducing inflammation in a subject in need thereof, comprising administering an effective dose of any one or more of the Veillonella dispar strains disclosed herein; any one or more of the Veillonella parvula strains disclosed herein; any one or more of the Veillonella atypica strains disclosed herein; and/or any one or more of the compositions disclosed herein (comprising one or more of the aforementioned bacteria) to the subject. The disclosure provides methods of enhancing recovery from physical exercise in a subject in need thereof, comprising administering an effective dose of any one or more of the Veillonella dispar strains disclosed herein; any one or more of the Veillonella parvula strains disclosed herein; any one or more of the Veillonella atypica strains disclosed herein; and/or any one or more of the compositions disclosed herein (comprising one or more of the aforementioned bacteria) to the subject.

The disclosure further provides methods of increasing muscle mass and/or muscular strength of a subject in need thereof, comprising administering an effective dose of any one or more of the Veillonella dispar strains disclosed herein; any one or more of the Veillonella parvula strains disclosed herein; any one or more of the Veillonella atypica strains disclosed herein; and/or any one or more of the compositions disclosed herein (comprising one or more of the aforementioned bacteria) to the subject. The disclosure further provides methods of preventing the loss of muscle mass of a subject in need thereof, comprising administering an effective dose of any one or more of the Veillonella dispar strains disclosed herein; any one or more of the Veillonella parvula strains disclosed herein; any one or more of the Veillonella atypica strains disclosed herein; and/or any one or more of the compositions disclosed herein (comprising one or more of the aforementioned bacteria) to the subject.

In some embodiments, the administration is via oral, enteric, gastrointestinal, or rectal route. In some embodiments, the subject is a human subject. In some embodiments, the dose is in the range of about 10⁴ CFU to about 10¹⁶ CFU. In some embodiments, the dose is in the range of about 10⁹ to about 10¹¹ CFU. In some embodiments, the dose is in the range of about 5×10⁹ CFU to about 10¹⁰ CFU.

### BRIEF DESCRIPTION OF THE DRAWINGS

FIG. 1A shows the sequence of 16S rRNA gene identified in the Veillonella dispar strain (SEQ ID NO: 1), and the variable and constant regions within the gene; FIG. 1B shows the sequence of 16S rRNA gene identified in the Veillonella parvula strain (SEQ ID NO: 11), and the variable and constant regions within the gene; and FIG. 1C shows the sequence of 16S rRNA gene identified in the Veillonella atypica strain (SEQ ID NO: 21), and the variable and constant regions within the gene. The variable regions (VR1 through VR9) are indicated by sequences that are italicized and underlined, while the constant regions (CR1 through CR10) are indicated by sequences that are in bold. The regions of the 16S rRNA genes are ordered in the following manner: 5′ CR1-VR1-CR2-VR2-CR3-VR3-CR4-VR4-CR5-VR5-CR6-VR6-CR7-VR7-CR8-VR8-CR9-VR9-CR10 3′.

FIG. 2 shows the concentration of lactate, acetate and propionate produced by Veillonella atypica having deposit accession number PTA-126860, and compositions comprising this Veillonella atypica strain and one or more lactate producing bacteria, as measured by Mass Spectrometry. Details of this experiment are provided in Example 2 and Example 3. “MRS” refers to the commercial medium that is typically used to grow the Lactobacillus and Bifidobacterium strains. “MRS lactate” refers to MRS medium supplemented with sodium lactate. L=lactate; A=acetate; P=propionate.

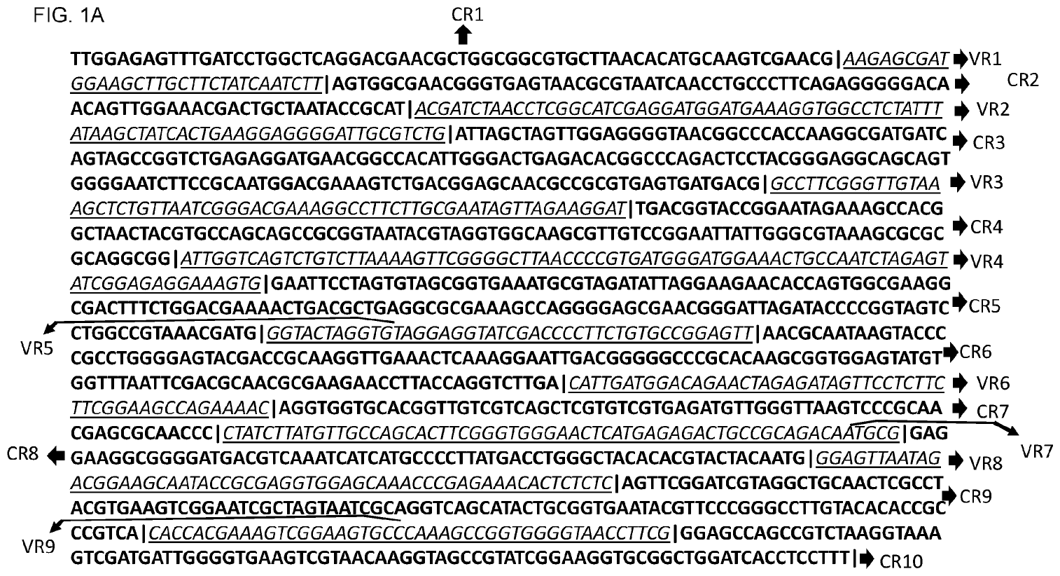

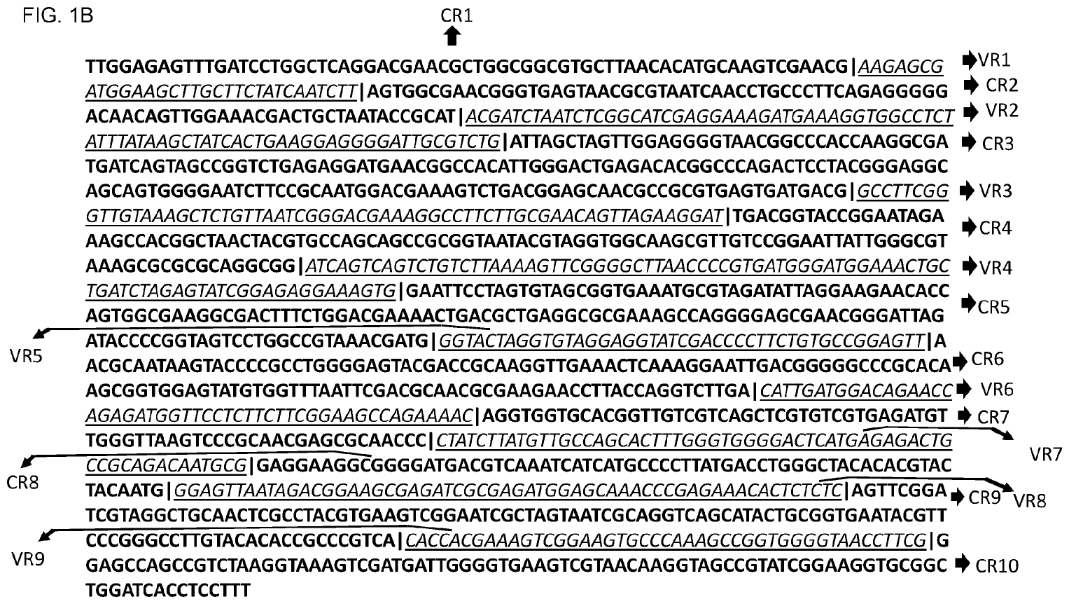

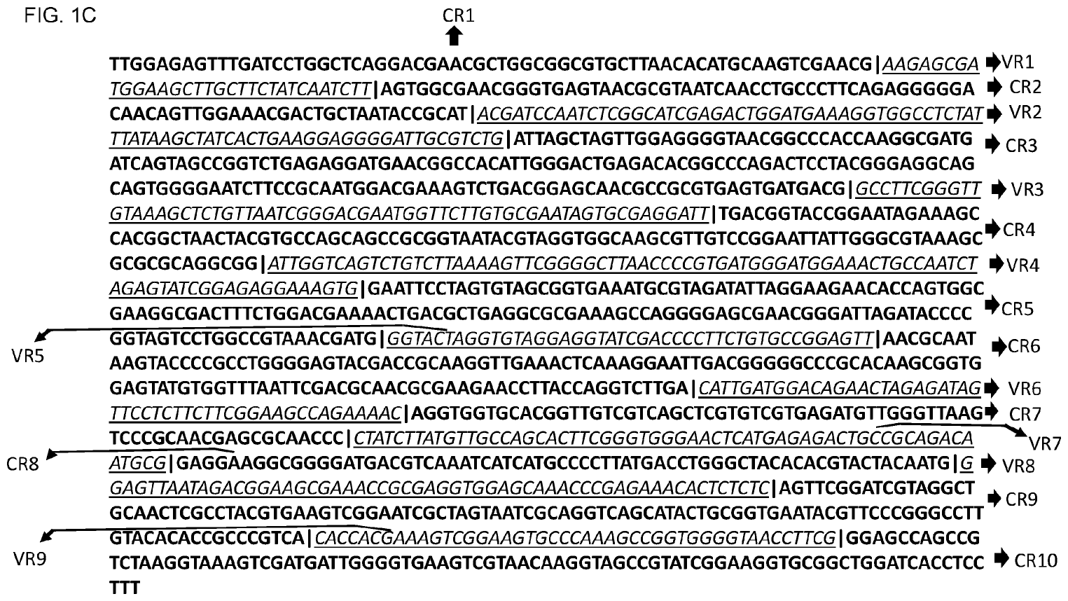

### DETAILED DESCRIPTION

### Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

As used herein, terms such as “a,” “an,” and “the” include singular and plural referents unless the context clearly demands otherwise.

As used herein, the term “about” when preceding a numerical value indicates the value plus or minus a range of 10%. For example, “about 100” encompasses 90 and 110.

As used herein, the term “subject” includes humans and other animals. Typically, the subject is a human. For example, the subject may be an adult or a teenager. In some embodiments, the adults are seniors about 65 years or older, or about 60 years or older. In some embodiments, the subject may be an animal which is being trained or is skilled in athletic activities, such as a horse. In some embodiments, the subject is an athlete, or a person training to be an athlete.

As used herein, the term “athlete” refers to a human subject who follows a regular exercise regimen. The term “regular exercise regimen” is not limited, and may be determined by a person of ordinary skill in the art, a physician or an athletic trainer. For instance, a regular exercise regimen may comprise performing a “physical exercise” at least once a week, for example twice a week, or thrice a week. As used herein, the term “non-athlete” refers to a human subject who does not follow a “regular exercise regimen”. In some embodiments, the athlete is a person trained and/or skilled in any form of physical exercise. In some embodiments, the athlete possesses enhanced physical strength, agility, endurance, speed and/or stamina as compared to the non-athlete. Methods to measure physical strength, agility, endurance, speed and/or stamina are commonly known, and may be chosen by one of ordinary skill in the art.

As used herein, the term “physical exercise” refers to any activity that involves physical exertion. In some embodiments, physical exercise enhances or maintains physical fitness, strengthens muscles and the cardiovascular system, promotes weight loss or maintenance, and/or enhances physical and/or mental health. Non-limiting examples of physical exercise include running, cycling, swimming, brisk walking, skipping rope, rowing, hiking, dancing, playing tennis, continuous training, long distance running, push-ups, pull-ups, lunges, squats, bench press, weight training, functional training, eccentric training, interval training, sprinting, and high-intensity interval training.

As used herein, “athletic performance” or “exercise performance” refers to one or more objective factors related to a particular physical exercise for a subject. In some embodiments, the objective factor is measurable and defined, such as the distance of a run, the height of a jump, or the distance an object is thrown. An improvement of athletic performance comprises an improvement of one or more objective factors related to the particular physical exercise for the particular subject. For instance, for a race, an improvement of athletic performance may comprise an increase in the distance run, a decrease in the time taken to run a particular distance, or a combination thereof. In some embodiments, the objective factor is exercise endurance.

As used herein, “exercise endurance” (interchangeably referred to herein as “training endurance” and “performance endurance”) refers to an ability to perform a particular physical exercise over a given period of time, and/or perform a physical exercise of a particular strenuousness. In some embodiments, increasing exercise endurance comprises an increase in the time that a physical exercise is performed, and/or an increase in the strenuousness of the exercise until exhaustion time. As used herein, “exhaustion time” or “exhaustion point” refers to the endpoint of a period of time, or a particular strenuousness, beyond which a particular physical exercise cannot be performed by a subject, due to, for example, fatigue.

As used herein, “microbiome” refers to the collection of microorganisms that inhabit the digestive tract or gastrointestinal (GI) tract of an animal (including the GI tract of a human) and the microorganism's physical environment (i.e. the microbiome has a biotic and abiotic component). The microbiome is fluid and may be modulated by numerous naturally occurring and artificial conditions (e.g., change in diet, disease, antimicrobial agents, influx of additional microorganisms, etc.). The modulation or optimization or alteration or shifting of the gut microbiome achieved via administration of the microbial strains and/or compositions of the present disclosure, can take the form of: (a) changing the diversity; i.e., increasing or decreasing a particular Family, Genus, Species, strain or functional grouping of microbes (i.e. alteration of the biotic component of the microbiome); (b) increasing or decreasing ratios of a particular Family, Genus, Species, strain or functional grouping of microbes; (c) increasing or decreasing polyphenols, proteins and/or metabolic compounds such as lactic acid, lactates, propionic acid, propionates, acetic acid, or acetates (i.e. alteration of the abiotic components of the microbiome) and/or (d) changing the quality of the microbiome; such as, for example increasing the proportion of microbes associated with enhanced exercise endurance.

The terms “microorganism” or “microbe” in certain instances may refer to an organism of microscopic size, to a single-celled organism, and/or to any virus particle. The definition of microorganism used herein includes Bacteria, Archaea, single-celled Eukaryotes (protozoa, fungi, and ciliates), and viral agents.

As used herein, “athlete-associated gut microbe” refers to a Family, Genus, Species, strain or functional grouping of a microbe that inhabits the digestive tract or gastrointestinal (GI) tract of an athlete.

As used herein, an “effective dose” or “effective amount” refers to an amount of substance able to achieve a desired outcome; for example, an amount of any one of the microbial strains or compositions disclosed herein that is sufficient to affect a desired outcome, such as an improvement of athletic performance.

As used herein, “inflammation” refers to a complex biological response of body tissues to harmful stimuli, such as pathogens, damaged cells, or irritants, and is a protective response involving immune cells, blood vessels, and molecular mediators, giving rise to one or more signs of heat, pain, redness, swelling, and loss of function. In some embodiments, the inflammation may be associated with, promoted by, or caused by physical exercise.

As used herein, “recovery from physical exercise” refers to the process by which one or more parts of the body, such as muscles, recover from the effect of physical exercise. In some embodiments, “recovery from physical exercise” comprises an alleviation of inflammation associated with physical exercise. The recovery from physical exercise may be associated with or promoted by one or more of the following: rest, sleep, hydration, stretching, nutrition, and massage. In some embodiments, recovery from physical exercise comprises a decrease in the levels of lactic acid and/or lactates in the blood, and/or an increase in the levels of any one or more of the following: propionic acid, propionates, acetic acid, and acetates, in the blood. In some embodiments, “enhancing recovery from physical exercise” comprises a decrease in the time taken to recover from physical exercise.

As used herein, short chain fatty acids (SCFAs) refer to fatty acids with fewer than 6 carbon atoms. In some embodiments, SCFAs are generated in the gastrointestinal tract of a subject, for example, from the fermentation of indigestible foods by the gut microbiome. Non-limiting examples of SCFAs are formic acid, acetic acid, propionic acid, butyric acid, isobutyric acid, valeric acid, isovaleric acid and 2-methylbutanoic acid.

### Athlete-Associated Gut Microbes, and Compositions Comprising the Same

The disclosure provides microbes that inhabit the digestive tract or gastrointestinal (GI) tract of an athlete, referred to herein as athlete-associated gut microbes. In some embodiments, the athlete-associated gut microbes inhabit the ileum or colon of the athlete. In some embodiments, the athlete-associated gut microbes are capable of metabolizing lactic acid and/or lactates. In some embodiments, the athlete-associated gut microbes are capable of metabolizing lactic acid and/or lactates into products, such as, for example, short-chain fatty acids (SCFAs), such as acetate and propionate. Therefore, in some embodiments, the one or more athlete-associated gut microbes is capable of producing propionic acid, propionates, acetic acid and/or acetates. In some embodiments, the athlete-associated gut microbes comprise one or more enzymes required for the conversion of lactate into SCFAs, such as acetate and propionate. In some embodiments, the athlete-associated gut microbes comprise at least one gene encoding at least one enzyme of the methylmalonyl-CoA pathway. In some embodiments, the athlete-associated gut microbes comprise one or more genes encoding all the enzymes of the methylmalonyl-CoA pathway. In some embodiments, the athlete-associated gut microbes comprise succinate-CoA transferase. Without being bound by a theory, it is thought that succinate-CoA transferase contributes to the conversion of lactate into SCFAs and propionate.

In some embodiments, the athlete-associated gut microbes are recombinant microbes, which have been engineered to metabolize lactic acid and/or lactates into products, such as, for example, short-chain fatty acids (SCFAs), such as acetate and propionate. In some embodiments, the recombinant athlete-associated gut microbes are engineered to encode one or more enzymes of the methylmalonyl-CoA pathway.

In some embodiments, the athlete-associated gut microbes are associated with, promote or cause enhanced exercise endurance, improved athletic performance, reduced inflammation, enhanced recovery from physical exercise, or any combination thereof in the athlete. In some embodiments, the population (or number or abundance) of one or more athlete-associated gut microbe is different (for example, higher or lower) in an athlete, as compared to a non-athlete. In some embodiments, the proportion of one or more athlete-associated gut microbe among the total number of gut microbes is different (higher or lower) in an athlete, as compared to a non-athlete.

In some embodiments, the activity of one or more athlete-associated gut microbes to metabolize lactic acid and/or lactates is higher in the athlete, as compared to a non-athlete. In some embodiments, the activity of one or more athlete-associated gut microbes to produce propionic acid, propionates, acetic acid and/or acetates is higher, as compared to a non-athlete. In some embodiments, any one or more of the microbial strains disclosed herein are resistant to gastric and bile acids. In some embodiments, any one or more of the microbial strains disclosed herein are capable of competing with pathogens for adhesion sites in the gut tissue. In some embodiments, any one or more of the microbial strains disclosed herein are capable of producing pathogen inhibitory substances, e.g. bacteriocidins and organic acids.

In some embodiments, the athlete-associated gut microbe comprises a microbial strain belonging to the genus Veillonella. In some embodiments, the microbial strain belonging to the genus Veillonella is an isolated and purified strain. In some embodiments, the microbial strain belonging to the genus Veillonella comprises a 16S ribosomal RNA (rRNA) gene comprising a nucleic acid sequence comprising at least one variable region. In some embodiments, the microbial strain belonging to the genus Veillonella comprises a 16S rRNA gene comprising a nucleic acid sequence comprising at least one constant region. In some embodiments, the at least one variable region (VR) is selected from the group consisting of: VR1, VR2, VR3, VR4, VR5, VR6, VR7, VR8, VR9, and any combination thereof. In some embodiments, the at least one constant region (CR) selected from the group consisting of: CR1, CR2, CR3, CR4, CR5, CR6, CR7, CR8, CR9, CR10, and any combination thereof.

In some embodiments, the microbial strain belonging to the genus Veillonella comprises a 16S rRNA gene comprising a nucleic acid sequence comprising the following variable regions: VR1, VR2, VR3, VR4, VR5, VR6, VR7, VR8, and VR9, and the following constant regions: CR1, CR2, CR3, CR4, CR5, CR6, CR7, CR8, CR9, CR10. In some embodiments, the VRs and CRs are ordered as shown below: 5′ CR1-VR1-CR2-VR2-CR3-VR3-CR4-VR4-CR5-VR5-CR6-VR6-CR7-VR7-CR8-VR8-CR9-VR9-CR10 3′

(a) Athlete-Associated Veillonella dispar Strains

In some embodiments, the athlete-associated gut microbe comprises a strain of Veillonella dispar. In some embodiments, the VR1 of Veillonella dispar comprises a nucleic acid sequence with at least about 80% sequence identity to SEQ ID NO: 2, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 2, including all subranges and values that lie therebetween.

In some embodiments, the VR2 of Veillonella dispar comprises a nucleic acid sequence with at least about 80% sequence identity to SEQ ID NO: 3, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 3, including all subranges and values that lie therebetween.

In some embodiments, the VR3 of Veillonella dispar comprises a nucleic acid sequence with at least about 80% sequence identity to SEQ ID NO: 4, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 4, including all subranges and values that lie therebetween.

In some embodiments, the VR4 of Veillonella dispar comprises a nucleic acid sequence with at least about 80% sequence identity to SEQ ID NO: 5, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 5, including all subranges and values that lie therebetween.

In some embodiments, the VR5 of Veillonella dispar comprises a nucleic acid sequence with at least about 80% sequence identity to SEQ ID NO: 6, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 6, including all subranges and values that lie therebetween.

In some embodiments, the VR6 of Veillonella dispar comprises a nucleic acid sequence with at least about 80% sequence identity to SEQ ID NO: 7, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 7, including all subranges and values that lie therebetween.

In some embodiments, the VR7 of Veillonella dispar comprises a nucleic acid sequence with at least about 80% sequence identity to SEQ ID NO: 8, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 8, including all subranges and values that lie therebetween.

In some embodiments, the VR8 of Veillonella dispar comprises a nucleic acid sequence with at least about 80% sequence identity to SEQ ID NO: 9, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 9, including all subranges and values that lie therebetween.

In some embodiments, the VR9 of Veillonella dispar comprises a nucleic acid sequence with at least about 80% sequence identity to SEQ ID NO: 10, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 10, including all subranges and values that lie therebetween.

In some embodiments, the CR1 of Veillonella dispar comprises a nucleic acid sequence with at least about 80% sequence identity to SEQ ID NO: 31, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 31, including all subranges and values that lie therebetween.

In some embodiments, the CR2 of Veillonella dispar comprises a nucleic acid sequence with at least about 80% sequence identity to SEQ ID NO: 32, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 32, including all subranges and values that lie therebetween.

In some embodiments, the CR3 of Veillonella dispar comprises a nucleic acid sequence with at least about 80% sequence identity to SEQ ID NO: 33, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 33, including all subranges and values that lie therebetween.

In some embodiments, the CR4 of Veillonella dispar comprises a nucleic acid sequence with at least about 80% sequence identity to SEQ ID NO: 34, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 34, including all subranges and values that lie therebetween.

In some embodiments, the CR5 of Veillonella dispar comprises a nucleic acid sequence with at least about 80% sequence identity to SEQ ID NO: 35, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 35, including all subranges and values that lie therebetween.

In some embodiments, the CR6 of Veillonella dispar comprises a nucleic acid sequence with at least about 80% sequence identity to SEQ ID NO: 36, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 36, including all subranges and values that lie therebetween.

In some embodiments, the CR7 of Veillonella dispar comprises a nucleic acid sequence with at least about 80% sequence identity to SEQ ID NO: 37, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 37, including all subranges and values that lie therebetween.

In some embodiments, the CR8 of Veillonella dispar comprises a nucleic acid sequence with at least about 80% sequence identity to SEQ ID NO: 38, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 38, including all subranges and values that lie therebetween.

In some embodiments, the CR9 of Veillonella dispar comprises a nucleic acid sequence with at least about 80% sequence identity to SEQ ID NO: 39, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 39, including all subranges and values that lie therebetween.

In some embodiments, the CR10 of Veillonella dispar comprises a nucleic acid sequence with at least about 80% sequence identity to SEQ ID NO: 40, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 40, including all subranges and values that lie therebetween.

In some embodiments, the strain of Veillonella dispar comprises a 16S rRNA gene comprising a nucleic acid sequence comprising at least about 80% sequence identity to SEQ ID NO: 1, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 1, including all subranges and values that lie therebetween.

The disclosure further provides an isolated and purified Veillonella dispar having deposit accession number PTA-126861, or a strain having all of the identifying characteristics of Veillonella dispar PTA-126861, or a mutant thereof.

(b) Athlete-Associated Veillonella parvula Strains

In some embodiments, the athlete-associated gut microbe comprises a strain of Veillonella parvula. In some embodiments, the VR1 of Veillonella parvula comprises a nucleic acid sequence with at least about 80% sequence identity to SEQ ID NO: 12, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 12, including all subranges and values that lie therebetween.

In some embodiments, the VR2 of Veillonella parvula comprises a nucleic acid sequence with at least about 80% sequence identity to SEQ ID NO: 13, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 13, including all subranges and values that lie therebetween.

In some embodiments, the VR3 of Veillonella parvula comprises a nucleic acid sequence with at least about 80% sequence identity to SEQ ID NO: 14, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 14, including all subranges and values that lie therebetween.

In some embodiments, the VR4 of Veillonella parvula comprises a nucleic acid sequence with at least about 80% sequence identity to SEQ ID NO: 15, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 15, including all subranges and values that lie therebetween.

In some embodiments, the VR5 of Veillonella parvula comprises a nucleic acid sequence with at least about 80% sequence identity to SEQ ID NO: 16, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 16, including all subranges and values that lie therebetween.

In some embodiments, the VR6 of Veillonella parvula comprises a nucleic acid sequence with at least about 80% sequence identity to SEQ ID NO: 17, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 17, including all subranges and values that lie therebetween.

In some embodiments, the VR7 of Veillonella parvula comprises a nucleic acid sequence with at least about 80% sequence identity to SEQ ID NO: 18, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 18, including all subranges and values that lie therebetween.

In some embodiments, the VR8 of Veillonella parvula comprises a nucleic acid sequence with at least about 80% sequence identity to SEQ ID NO: 19, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 19, including all subranges and values that lie therebetween.

In some embodiments, the VR9 of Veillonella parvula comprises a nucleic acid sequence with at least about 80% sequence identity to SEQ ID NO: 20, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 20, including all subranges and values that lie therebetween.

In some embodiments, the CR1 of Veillonella parvula comprises a nucleic acid sequence with at least about 80% sequence identity to SEQ ID NO: 41, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 41, including all subranges and values that lie therebetween.

In some embodiments, the CR2 of Veillonella parvula comprises a nucleic acid sequence with at least about 80% sequence identity to SEQ ID NO: 42, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 42, including all subranges and values that lie therebetween.

In some embodiments, the CR3 of Veillonella parvula comprises a nucleic acid sequence with at least about 80% sequence identity to SEQ ID NO: 43, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 43, including all subranges and values that lie therebetween.

In some embodiments, the CR4 of Veillonella parvula comprises a nucleic acid sequence with at least about 80% sequence identity to SEQ ID NO: 44, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 44, including all subranges and values that lie therebetween.

In some embodiments, the CR5 of Veillonella parvula comprises a nucleic acid sequence with at least about 80% sequence identity to SEQ ID NO: 45, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 45, including all subranges and values that lie therebetween.

In some embodiments, the CR6 of Veillonella parvula comprises a nucleic acid sequence with at least about 80% sequence identity to SEQ ID NO: 46, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 46, including all subranges and values that lie therebetween.

In some embodiments, the CR7 of Veillonella parvula comprises a nucleic acid sequence with at least about 80% sequence identity to SEQ ID NO: 47, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 47, including all subranges and values that lie therebetween.

In some embodiments, the CR8 of Veillonella parvula comprises a nucleic acid sequence with at least about 80% sequence identity to SEQ ID NO: 48, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 48, including all subranges and values that lie therebetween.

In some embodiments, the CR9 of Veillonella parvula comprises a nucleic acid sequence with at least about 80% sequence identity to SEQ ID NO: 49, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 49, including all subranges and values that lie therebetween.

In some embodiments, the CR10 of Veillonella parvula comprises a nucleic acid sequence with at least about 80% sequence identity to SEQ ID NO: 50, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 50, including all subranges and values that lie therebetween.

In some embodiments, the strain of Veillonella parvula comprises a 16S rRNA gene comprising a nucleic acid sequence comprising at least about 80% sequence identity to SEQ ID NO: 11, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 11, including all subranges and values that lie therebetween.

In addition, the disclosure provides an isolated and purified Veillonella parvula having deposit accession number PTA-126859, or a strain having all of the identifying characteristics of Veillonella parvula PTA-126859, or a mutant thereof.

(c) Athlete-Associated Veillonella atypica Strains

In some embodiments, the athlete-associated gut microbe comprises a strain of Veillonella atypica. In some embodiments, the VR1 of Veillonella atypica comprises a nucleic acid sequence with at least about 80% sequence identity to SEQ ID NO: 22, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 22, including all subranges and values that lie therebetween.

In some embodiments, the VR2 of Veillonella atypica comprises a nucleic acid sequence with at least about 80% sequence identity to SEQ ID NO: 23, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 23, including all subranges and values that lie therebetween.

In some embodiments, the VR3 of Veillonella atypica comprises a nucleic acid sequence with at least about 80% sequence identity to SEQ ID NO: 24, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 24, including all subranges and values that lie therebetween.

In some embodiments, the VR4 of Veillonella atypica comprises a nucleic acid sequence with at least about 80% sequence identity to SEQ ID NO: 25, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 25, including all subranges and values that lie therebetween.

In some embodiments, the VR5 of Veillonella atypica comprises a nucleic acid sequence with at least about 80% sequence identity to SEQ ID NO: 26, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 26, including all subranges and values that lie therebetween.

In some embodiments, the VR6 of Veillonella atypica comprises a nucleic acid sequence with at least about 80% sequence identity to SEQ ID NO: 27, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 27, including all subranges and values that lie therebetween.

In some embodiments, the VR7 of Veillonella atypica comprises a nucleic acid sequence with at least about 80% sequence identity to SEQ ID NO: 28, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 28, including all subranges and values that lie therebetween.

In some embodiments, the VR8 of Veillonella atypica comprises a nucleic acid sequence with at least about 80% sequence identity to SEQ ID NO: 29, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 29, including all subranges and values that lie therebetween.

In some embodiments, the VR9 of Veillonella atypica comprises a nucleic acid sequence with at least about 80% sequence identity to SEQ ID NO: 30, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 30, including all subranges and values that lie therebetween.

In some embodiments, the CR1 of Veillonella atypica comprises a nucleic acid sequence with at least about 80% sequence identity to SEQ ID NO: 51, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 51, including all subranges and values that lie therebetween.

In some embodiments, the CR2 of Veillonella atypica comprises a nucleic acid sequence with at least about 80% sequence identity to SEQ ID NO: 52, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 52, including all subranges and values that lie therebetween.

In some embodiments, the CR3 of Veillonella atypica comprises a nucleic acid sequence with at least about 80% sequence identity to SEQ ID NO: 53, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 53, including all subranges and values that lie therebetween.

In some embodiments, the CR4 of Veillonella atypica comprises a nucleic acid sequence with at least about 80% sequence identity to SEQ ID NO: 54, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 54, including all subranges and values that lie therebetween.

In some embodiments, the CR5 of Veillonella atypica comprises a nucleic acid sequence with at least about 80% sequence identity to SEQ ID NO: 55, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 55, including all subranges and values that lie therebetween.

In some embodiments, the CR6 of Veillonella atypica comprises a nucleic acid sequence with at least about 80% sequence identity to SEQ ID NO: 56, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 56, including all subranges and values that lie therebetween.

In some embodiments, the CR7 of Veillonella atypica comprises a nucleic acid sequence with at least about 80% sequence identity to SEQ ID NO: 57, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 57, including all subranges and values that lie therebetween.

In some embodiments, the CR8 of Veillonella atypica comprises a nucleic acid sequence with at least about 80% sequence identity to SEQ ID NO: 58, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 58, including all subranges and values that lie therebetween.

In some embodiments, the CR9 of Veillonella atypica comprises a nucleic acid sequence with at least about 80% sequence identity to SEQ ID NO: 59, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 59, including all subranges and values that lie therebetween.

In some embodiments, the CR10 of Veillonella atypica comprises a nucleic acid sequence with at least about 80% sequence identity to SEQ ID NO: 60, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 60, including all subranges and values that lie therebetween.

In some embodiments, the strain of Veillonella atypica comprises a 16S rRNA gene comprising a nucleic acid sequence comprising at least about 80% sequence identity to SEQ ID NO: 21, for example, at least about 85%, at least about 90%, at least about 95%, at least about 96%, at least about 97%, at least about 97.5%, at least about 98%, at least about 98.1%, at least 98.2%, at least about 98.3%, at least about 98.4%, at least about 98.5%, at least about 98.6%, at least about 98.7%, at least about 98.8%, at least about 98.9%, at least about 99%, at least about 99.5%, or about 100% sequence identity to SEQ ID NO: 21, including all subranges and values that lie therebetween.

Also, the disclosure provides an isolated and purified Veillonella atypica having deposit accession number PTA-126860, or a strain having all of the identifying characteristics of Veillonella atypica PTA-126860, or a mutant thereof.

### (d) Compositions Comprising Athlete-Associated Gut Microbes Disclosed Herein

The disclosure further provides compositions comprising any one or more of the strains of Veillonella sp. disclosed herein. The disclosure provides compositions comprising any one or more of the strains of Veillonella dispar disclosed herein. The disclosure also provides compositions comprising any one or more of the strains of Veillonella parvula disclosed herein. The disclosure also provides compositions comprising any one or more of the strains of Veillonella atypica disclosed herein.

In some embodiments, the composition comprises a lactate-producing microbe. The lactate-producing microbe may be any microbe that is capable of producing lactate and/or lactic acid, such as, lactate-producing bacteria. In some embodiments, the composition comprises equal amounts of the Veillonella sp. strain, and one or more of the lactate-producing bacteria. In some embodiments, all the constituent bacteria in the composition are present in a 1:1 ratio.

In some embodiments, the amount or concentration of a microbe in the composition is a non-naturally occurring amount or concentration of the microbe. In some embodiments, the composition comprises a non-naturally occurring amount or concentration of each of the microbes in the composition. In some embodiments, the total amount or concentration of all the microbes in the composition is not found in nature. In some embodiments, the composition comprises a non-naturally occurring ratio of the different microbes in the composition.

In some embodiments, the microbes are found in a composition along with an ingredient, carrier, or other component that does not naturally occur with said microbes in their natural state. In embodiments, the edible compositions comprise microbes in combination with a food ingredient that is not found with said microbes in their natural state. In aspects, the compositions taught here have markedly different characteristics compared to the microbes found in isolation in nature. In aspects, the microbial compositions are synergistic. In aspects, the disclosure teaches kits, which comprise microbes of the disclosure, along with a suitable packaging that protects the microbes from external stressors. In aspects, kits of the disclosure may comprise directions for use and/or administration of the packaged microbes. Thus, the disclosure teaches packaged microbes and instructions for administration.

Non-limiting examples of lactate-producing bacteria include bacteria belonging to the genus Lactobacillus, Bifidobacterium, Leuconostoc, Pediococcus, Lactococcus, Streptococcus, Aerococcus, Carnobacterium, Enterococcus, Oenococcus, Sporolactobacillus, Tetragenococcus, Vagococcus, and Weissella. In some embodiments, the lactate-producing bacteria belongs to the genus Lactobacillus. In some embodiments, the lactate-producing bacteria is Lactobacillus plantarum, Lactobacillus acidophilus, Lactobacillus rhamnosus, or any combination thereof. In some embodiments, the lactate-producing bacteria belongs to the genus Bifidobacterium. In some embodiments, the lactate-producing bacteria is Bifidobacterium longum, Bifidobacterium lactis or a combination thereof. Bifidobacterium lactis may be interchangeably referred to as, Bifidobacterium animalis lactis or Bifidobacterium animalis subsp. lactis.

Thus, the disclosure provides compositions comprising any one or more of the microbial strains disclosed herein, and any one or more of the following microbes: Lactobacillus plantarum, Lactobacillus acidophilus, Lactobacillus rhamnosus, and Bifidobacterium longum, Bifidobacterium lactis.

In some embodiments, the compositions comprise the Veillonella atypica having deposit accession number PTA-126860, or a strain having all of the identifying characteristics of Veillonella atypica PTA-126860, or a mutant thereof, and any one or more of the following microbes: Lactobacillus plantarum, Lactobacillus acidophilus, Lactobacillus rhamnosus, Bifidobacterium longum, and Bifidobacterium lactis. In some embodiments, the compositions comprise the Veillonella atypica having deposit accession number PTA-126860 and Lactobacillus plantarum. In some embodiments, the compositions comprise the Veillonella atypica having deposit accession number PTA-126860 and Lactobacillus acidophilus. In some embodiments, the compositions comprise the Veillonella atypica having deposit accession number PTA-126860 and Lactobacillus rhamnosus. In some embodiments, the compositions comprise the Veillonella atypica having deposit accession number PTA-126860 and Bifidobacterium longum. In some embodiments, the compositions comprise the Veillonella atypica having deposit accession number PTA-126860 and Bifidobacterium lactis.

In some embodiments, the compositions comprise the Veillonella parvula having deposit accession number PTA-126859, or a strain having all of the identifying characteristics of Veillonella parvula PTA-126859, or a mutant thereof, and any one or more of the following microbes: Lactobacillus plantarum, Lactobacillus acidophilus, Lactobacillus rhamnosus, Bifidobacterium longum, and Bifidobacterium lactis. In some embodiments, the compositions comprise the Veillonella parvula having deposit accession number PTA-126859 and Lactobacillus plantarum. In some embodiments, the compositions comprise the Veillonella parvula having deposit accession number PTA-126859 and Lactobacillus acidophilus. In some embodiments, the compositions comprise the Veillonella parvula having deposit accession number PTA-126859 and Lactobacillus rhamnosus. In some embodiments, the compositions comprise the Veillonella parvula having deposit accession number PTA-126859 and Bifidobacterium longum. In some embodiments, the compositions comprise the Veillonella parvula having deposit accession number PTA-126859 and Bifidobacterium lactis.

In some embodiments, the compositions comprise the Veillonella dispar having deposit accession number PTA-126861, or a strain having all of the identifying characteristics of Veillonella dispar PTA-126861, or a mutant thereof, and any one or more of the following microbes: Lactobacillus plantarum, Lactobacillus acidophilus, Lactobacillus rhamnosus, Bifidobacterium longum, and Bifidobacterium lactis. In some embodiments, the compositions comprise the Veillonella dispar having deposit accession number PTA-126861 and Lactobacillus plantarum. In some embodiments, the compositions comprise the Veillonella dispar having deposit accession number PTA-126861 and Lactobacillus acidophilus. In some embodiments, the compositions comprise the Veillonella dispar having deposit accession number PTA-126861 and Lactobacillus rhamnosus. In some embodiments, the compositions comprise the Veillonella dispar having deposit accession number PTA-126861 and Bifidobacterium longum. In some embodiments, the compositions comprise the Veillonella dispar having deposit accession number PTA-126861 and Bifidobacterium lactis.

The disclosure provides compositions comprising: any one or more of the Veillonella sp. strains disclosed herein, and any one or more of the following strains: (a) an isolated and purified Lactobacillus plantarum comprising a 16S rRNA nucleic acid sequence that is at least 97% identical to SEQ ID NO: 61; (b) an isolated and purified Lactobacillus acidophilus comprising a 16S rRNA nucleic acid sequence that is at least 97% identical to SEQ ID NO: 62; (c) an isolated and purified Lactobacillus rhamnosus comprising a 16S rRNA nucleic acid sequence that is at least 97% identical to SEQ ID NO: 63; (d) an isolated and purified Bifidobacterium longum comprising a 16S rRNA nucleic acid sequence that is at least 97% identical to SEQ ID NO: 64; (e) an isolated and purified Bifidobacterium lactis comprising a 16S rRNA nucleic acid sequence that is at least 97% identical to SEQ ID NO: 65; and (f) an isolated and purified Lactobacillus paracasei comprising a 16S rRNA nucleic acid sequence that is at least 97% identical to SEQ ID NO: 66.

For instance, in some embodiments, the compositions comprise: (a) an isolated and purified Veillonella atypica having deposit accession number PTA-126860, or a strain having all of the identifying characteristics of Veillonella atypica PTA-126860, or a mutant thereof, (b) an isolated and purified Lactobacillus plantarum comprising a 16S rRNA nucleic acid sequence that is at least 97% identical to SEQ ID NO: 61; (c) an isolated and purified Lactobacillus acidophilus comprising a 16S rRNA nucleic acid sequence that is at least 97% identical to SEQ ID NO: 62; (d) an isolated and purified Lactobacillus rhamnosus comprising a 16S rRNA nucleic acid sequence that is at least 97% identical to SEQ ID NO: 63; (e) an isolated and purified Bifidobacterium longum comprising a 16S rRNA nucleic acid sequence that is at least 97% identical to SEQ ID NO: 64; and (f) an isolated and purified Bifidobacterium lactis comprising a 16S rRNA nucleic acid sequence that is at least 97% identical to SEQ ID NO: 65.

The disclosure further provides compositions comprising: any one or more of the Veillonella sp. strains disclosed herein, and any one or more of the following strains: (a) an isolated and purified Lactobacillus plantarum comprising a 16S rRNA nucleic acid sequence that is at least 97% identical to SEQ ID NO: 61; (b) an isolated and purified Lactobacillus acidophilus comprising a 16S rRNA nucleic acid sequence that is at least 97% identical to SEQ ID NO: 62; (c) an isolated and purified Lactobacillus rhamnosus comprising a 16S rRNA nucleic acid sequence that is at least 97% identical to SEQ ID NO: 63; and (d) an isolated and purified Lactobacillus paracasei comprising a 16S rRNA nucleic acid sequence that is at least 97% identical to SEQ ID NO: 66.

For instance, in some embodiments, the compositions comprise an isolated and purified Veillonella atypica having deposit accession number PTA-126860, or a strain having all of the identifying characteristics of Veillonella atypica PTA-126860, or a mutant thereof, an isolated and purified Lactobacillus plantarum comprising a 16S rRNA nucleic acid sequence that is at least 97% identical to SEQ ID NO: 61; an isolated and purified Lactobacillus acidophilus comprising a 16S rRNA nucleic acid sequence that is at least 97% identical to SEQ ID NO: 62; and an isolated and purified Lactobacillus rhamnosus comprising a 16S rRNA nucleic acid sequence that is at least 97% identical to SEQ ID NO: 63.

Without being bound by any one theory, it is thought that in compositions comprising any one or more of the athlete-associated gut microbes disclosed herein, and a lactate-producing microbe, the lactate produced by the lactate-producing microbe is utilized by the athlete-associated gut microbe to produce acetate and/or propionate. Thus, in some embodiments, the compositions comprising any one or more of the athlete-associated gut microbes disclosed herein, and a lactate-producing microbe are capable of producing more short chain fatty acids (SCFAs), such as, acetate than the athlete-associated gut microbe alone. In some embodiments, the increased acetate produced by the composition is due to a synergy between the athlete-associated gut microbe and the lactate-producing microbe. In some embodiments, the lactate-producing microbe does not produce propionate and/or acetate.

In some embodiments, the compositions comprising any one or more of the athlete-associated gut microbes disclosed herein, and a lactate-producing microbe are capable of producing at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, or about 20 fold, including all subranges and values that lie therebetween) more acetate than the athlete-associated gut microbe alone.

In some embodiments, the compositions comprising any one or more of the athlete-associated gut microbes disclosed herein, and a lactate-producing microbe are capable of producing at least about 1% (for example, about 2%, about 5%, about 10%, about 15%, about 20%, about 25%, about 30%, about 40%, about 50%, about 60%, about 70%, about 80%, about 90%, or about 100%, including all subranges and values that lie therebetween) more acetate than either the athlete-associated gut microbe alone.

In some embodiments, the compositions comprising any one or more of the athlete-associated gut microbes disclosed herein, and a lactate-producing microbe produce less propionate than the athlete-associated gut microbe alone. In some embodiments, the compositions comprising any one or more of the athlete-associated gut microbes disclosed herein, and a lactate-producing microbe produce at least about 1% (for example, about 2%, about 5%, about 10%, about 15%, about 20%, about 25%, about 30%, about 40%, about 50%, about 60%, about 70%, about 80%, about 90%, or about 100%, including all subranges and values that lie therebetween) less propionate than the athlete-associated gut microbe alone.

In some embodiments, the compositions comprising any one or more of the athlete-associated gut microbes disclosed herein, and a lactate-producing microbe are capable of producing more acetate and less propionate than the athlete-associated gut microbe alone.

In some embodiments, the compositions disclosed herein comprise an additive that is capable of enhancing one or more functional properties of the Veillonella strain comprised in the composition. For instance, the additive may increase the level of short chain fatty acids (SCFAs), such as, acetate and/or propionate, produced by the Veillonella strain. In some embodiments, the additive that is capable of increasing the level of acetate and/or propionate produced by the Veillonella strain is a lactate. In some embodiments, the lactate is lactate ion or lactic acid. In some embodiments, the lactate is sodium lactate, magnesium lactate, calcium lactate, manganese lactate, potassium lactate, cobalt lactate or iron lactate. Without being bound by a theory, it is thought that Veillonella is capable of metabolizing lactates, which in turn might contribute to an increase in acetate and/or propionate production. Further, lactate salts, such as, sodium lactate, magnesium lactate, calcium lactate, and potassium lactate can also act as electrolytes and promote body hydration. In some embodiments, the additive is a nitrate, such as sodium nitrate. Without being bound by any one theory, it is thought that the Veillonella strain metabolizes nitrate to produce nitric oxide, which also has beneficial properties in increasing endurance.

In some embodiments, the compositions comprise one or more additives, such as, dietary supplements. In some embodiments, the dietary supplement is a probiotic or prebiotic known to one of skill in the art. In some embodiments, the dietary supplement is one or more non-digestible dietary supplements, which modifies the balance of the intestinal micro flora, stimulates the growth and/or activity of beneficial microorganisms and suppresses potentially deleterious microorganisms. Non-limiting examples of dietary supplements include oligosaccharides (fructo-oligosaccharides, galacto-oligosaccharides), inulin, lactulose, lactitol and select bacterial strains that produce nutrients that promote the growth of beneficial bacteria, such as within the intestinal tract. In some embodiments, the dietary supplement is a dietary fiber, such as, for example, cellulose, chitin, hemicellulose, lignin, xanthan gum, fructans, pectin, alginates (e.g., sodium alginate), agar, carrageen, raffinose and polydextrose. In some embodiments, the dietary supplement is an amino acid, or a protein.

Any one of the compositions disclosed herein may further comprise any conventional food supplement fillers and extenders such as, for example, a flour, a binder, a nutraceutical compound or formulation, or a nutrient such as, an amino acid (e.g., branched chain amino acids, such as, leucine, isoleucine, and valine), a vitamin (e.g., vitamin D, folate, vitamin B-12), or a mineral (e.g., magnesium, calcium, zinc, iron). In some embodiments, the additive is an anti-oxidant (e.g., vitamin C, vitamin E, beta-carotene, carotenoids, selenium, manganese, lycopene, lutein and zeaxanthin), an anti-inflammatory substance (e.g., alpha-lipoic acid, curcumin, fish oil, ginger, resveratrol, spirulina) and/or an adaptogen (e.g., ginseng). In some embodiments, the additive is maltodextrin.

In some embodiments, the additive is capable of enhancing exercise endurance in a subject when administered to the subject. Non-limiting examples of such additives include caffeine, beta-alanine, carnosine, anserine, carbonate, carbohydrates, electrolytes, glycerol, nitrates, citrulline, and arginine. In some embodiments, the additive is capable of improving the athletic performance of a subject when administered to the subject. Non-limiting examples of such additives include creatine, protein, amino acids, essential amino acids (EAA), branched chain amino acids, beta-hydroxy beta-methylbutyric acid (HMB), 2-Hydroxyisocaproic acid (HICA), adenosine 5′-triphosphate (ATP), phosphatidic acid, phosphatidylserine, arachidonic acid, taurine, carnitine, and medium chain fatty acids. In some embodiments, the additive is capable of reducing inflammation in a subject when administered to the subject. Non-limiting examples of such additives include curcumin and omega-3 fatty acids. In some embodiments, the additive is capable of enhancing recovery from physical exercise in a subject when administered to the subject. Non-limiting examples of such additives include β-glucan, HMB, BCAAs, protein, curcumin, anti-oxidants, such as vitamin E, CoQ10, and alpha-lipoic acid.

In some embodiments, the additive is a vegetable extract or a fruit extract. In some embodiments, the fruit extract or vegetable extract naturally contains lactic acid. In some embodiments, the fruit extract is derived from Akebia fruit.

In some embodiments, the composition is a human edible formulation. In some embodiments, the composition is in a solid form (such as a lyophilized powder suitable for reconstitution), a liquid solution, suspension, emulsion, tablet, caplet, syrup, gummies, gels, pill, capsule, sustained release formulation, or powder. In some embodiments, the composition is a dry powder. In some embodiments, the composition is an encapsulated powder. In some embodiments, the composition is designed such that the microbe is not in contact with moisture and/or air. In some embodiments, delivery vehicles such as liposomes, nanocapsules, microparticles, microspheres, lipid particles, vesicles, and the like, may be used for the delivery of the microbial strains or compositions disclosed herein. In some embodiments, the composition is a human edible formulation (e.g., an organic human edible formulation), such as, for example, a protein powder, a protein supplement, a protein supplement shake, a protein supplement formed and packed as a bar, a beverage, a gel composition, a food composition, a freeze dried product or powder, a cereal bar, an energy bar (e.g., nutritional supplement energy bar, an organic energy bar), a fruit-based food bar (e.g., an organic fruit-based food bar), a fruit-based meal replacement bar, a nut-based food bar, a nut-based snack bar, a yogurt, a yoghurt drink, a yoghurt-based beverage, a kefir, a electrolyte replacement solution, a sports drink (e.g., sports drinks containing electrolytes, vitamins and probiotics), a non-alcoholic water-based beverage (e.g., one containing electrolytes, vitamins and probiotics); a protein-enriched sports beverage, a non-alcoholic water-based beverage (e.g., one containing protein), a caplet, a chewable formulation, granule, a dissolvable formulation, and/or fermented, dairy product, such as a chilled dairy product, or a fresh cheese. For instance, in some embodiments, a dry powdered form of the microbe or composition disclosed herein may be spread over a cereal bar, an energy bar, or any human edible formulation described herein. In some embodiments, an encapsulated form of the microbe or composition disclosed herein may be included in a liquid edible formulation, such as, a beverage. In some embodiments, the composition is a probiotic composition for use as ingredient for food or beverage, such as probiotic bacteria or probiotic bacterial cultures. In some embodiments, the composition is a probiotic supplement or nutritional supplement, or used in the manufacture of a probiotic supplement or nutritional supplement. In some embodiments, the composition is in the form of a beverage containing probiotics for use as a nutritional supplement. In some embodiments, the composition is a nutritional probiotic supplement in the form of capsules, tablets, caplets, powder, syrups, gummies and gels.

Any one of the compositions disclosed herein may further comprise a pharmaceutically acceptable carrier. By “pharmaceutically acceptable” it is meant a material that is not toxic or otherwise undesirable, i.e., the material may be administered to a subject without causing any significant undesirable biological effects. Pharmaceutically acceptable carriers or excipients are described, for example, in Remington's Pharmaceutical Sciences, Mack Publishing Co. (A R. Gennaro edit. 1985), Handbook of Pharmaceutical Additives, 2nd Edition (eds. M. Ash and I. Ash), 2001 (Synapse Information Resources, Inc., Endicott, N.Y., USA), and Handbook of Pharmaceutical Excipients, 2nd edition, 1994, the contents of each of which is herein incorporated by reference in its entirety for all purposes. In some embodiments, the compositions disclosed herein further comprise at least one pharmaceutically acceptable carrier, excipient, and/or vehicle, for example, solvents, buffers, solutions, dispersion media, coatings, antibacterial and antifungal agents, isotonic and absorption delaying agents. In some embodiments, the pharmaceutically acceptable carrier, excipient, and/or vehicle may comprise saline, buffered saline, dextrose, water, glycerol, sterile isotonic aqueous buffer, and combinations thereof. In some embodiments, the pharmaceutically acceptable carrier, excipient, and/or vehicle comprises phosphate buffered saline, sterile saline, lactose, sucrose, calcium phosphate, dextran, agar, pectin, peanut oil, sesame oil, pharmaceutical grades of mannitol, lactose, starch, magnesium stearate, sodium saccharine, cellulose, magnesium carbonate, polyol (e.g., glycerol, propylene glycol, and liquid polyethylene glycol, and the like) or suitable mixtures thereof. In some embodiments, the compositions disclosed herein further comprise minor amounts of emulsifying or wetting agents, or pH buffering agents.

In some embodiments, the compositions disclosed herein further comprise other conventional pharmaceutical ingredients, such as preservatives, or chemical stabilizers, such as chlorobutanol, potassium sorbate, sorbic acid, sulfur dioxide, propyl gallate, the parabens, ethyl vanillin, glycerin, phenol, parachlorophenol or albumin. In some embodiments, the compositions disclosed herein may further comprise antibacterial and antifungal agents, such as, parabens, chlorobutanol, phenol, sorbic acid or thimerosal; isotonic agents, such as, sugars or sodium chloride and/or agents delaying absorption, such as, aluminum monostearate and gelatin.

Any one of the compositions disclosed herein may further comprise a food carrier, or an edible carrier. As used herein, a “food carrier” or an “edible carrier” refer to materials that may be present in human edible formulations, such as food and beverages, and that are not toxic or otherwise undesirable, i.e., the food carrier and/or edible carrier may be consumed by a subject without resulting in any significant undesirable biological effects. Non-limiting examples of food carriers and/or edible carriers include, food acids (e.g. vinegar, citric acid, tartaric acid, malic acid), acidity regulators, anticaking agents, antifoaming agents, bulking agents, food coloring agents, color retention agents, emulsifiers, flavors, flavor enhancers, antioxidants, glazing agents, humectants, tracer gases, preservatives, stabilizers, sweeteners, and thickeners.

In some embodiments, the compositions disclosed herein further comprise one or more additives that promotes the growth of the one or more microbial strains in the composition and/or increases the ability of the microbial strain to be active in the gut. Non-limiting examples of such additives include cellobiose, maltose, mannose, salicine, trehalose, amygdalin, arabinose, melobiose, rhamnose and/or xylose. In some embodiments, the composition further comprises a drug, such as, acetaminophen, antibiotics such as an antibiotic selected from the group consisting of vancomycin, metronidazole, gentamicin, colistin, fidaxomicin, telavancin, oritavancin, dalbavancin and daptomycin, or any other beneficial substances. In some embodiments, the compositions disclosed herein further comprise pH encapsulated glucose, lipids or proteins, which are released at a pH in the range of about 1 to about 6. In some embodiments, the pH encapsulated glucose, lipids or proteins are released before the release of the one or more microbial strains in the composition. In some embodiments, the compositions disclosed herein enable the release of the athlete-associated gut microbes in the distal segments of the gastrointestinal tract, including the ileum and colon of the subject.

### Methods of Producing Compositions Comprising Athlete-Associated Microbial Strains

The disclosure provides methods of identifying and characterizing athlete-associated gut microbial strains. In some embodiments, the methods of identifying athlete-associated gut microbial strains, comprise isolating a stool sample of the athlete, and identifying the microbial strains that are present in the stool sample. In some embodiments, the identification step comprises isolating the DNA of microbial strains present in the stool sample, sequencing parts or whole of the 16S ribosomal DNA (rDNA) gene of the microbial strains present in the stool samples, and identifying the microbial strains present based on sequence alignment techniques.

The methods involved in the identification of microbes present in fecal sample, such as sequencing and sequence alignment, and subsequent characterization of the microbes are further described in “Probiotic formulations for improving athletic performance” WO 2017/180501 A1, published Oct. 19, 2017; Scheiman et al., “Meta-omics analysis of elite athletes identifies a performance-enhancing microbe that functions via lactate metabolism” Nature Medicine volume 25, July 2019, pages 1104-1109; and “Compositions and methods for enhancing exercise endurance” WO 2020/172604 A1, published Aug. 27, 2020, the contents of each of which is incorporated by reference herein in its entireties for all purposes.

Scheiman et al. observed that bacteria of the genus Veillonella are enriched in some athletes after exercise, and they isolated V. parvula, V. dispar and V. atypica from these athletes. They also saw that when V. atypica is inoculated into mice, exhaustive treadmill run time of the mice is increased, indicating that V. atypica can enhance physical performance. They also describe that Veillonella methylmalonyl-CoA pathway is overrepresented in athletes after exercise. Based on these results, Scheiman et al. concluded that bacteria of the genus Veillonella, such as V. atypica, can enhance athletic performance by increasing the levels of one or more performance-enhancing molecules, such as acetate and propionate, and potentially also by reducing the levels of lactate/lactic acid in the subject.

The present disclosure is aimed at finding additional, specific strains of V. parvula, V. dispar and V. atypica, which are genetically distinct from those of Scheiman et al, based on, for example, their 16S rRNA sequences. The disclosed strains, which were isolated from different athletes, are hypothesized to also be beneficial for improving athletic performance, reducing inflammatory cytokines, and enhancing endurance.

In some embodiments, the methods further comprise identifying microbial strains that are associated with the gut of a control human subject (such as, a non-athlete), comprising isolating a stool sample of the control subject, and identifying the microbial strains that are present in the stool sample, using the methods described above. In some embodiments, the methods further comprise comparing the diversity, proportion, and/or population of the gut microbial strains identified in an athlete with the diversity, proportion, and/or population of the gut microbial strains identified in a non-athlete.

In some embodiments, the methods further comprise comparing the lactate or lactic acid metabolizing activity of the gut microbial strains identified in an athlete with the lactate or lactic acid metabolizing activity of the gut microbial strains identified in a non-athlete. In some embodiments, the methods further comprise comparing the propionate or propionic acid producing activity of the gut microbial strains identified in an athlete with the propionate or propionic acid producing activity of the gut microbial strains identified in a non-athlete. The metabolic activity of the microbial strains, for example the lactate or lactic acid metabolizing activity, or the propionate, propionic acid, acetic acid and/or acetate-producing activity, may be determined using approaches, such as mass spectrometry (MS) of the media used to culture the microbial strains.

The athlete-associated microbial strains disclosed herein may be grown for commercial purposes using methods described herein. Any suitable medium, such as medium comprising a carbon-based substrate and/or a carbon based energy source may be used. In some embodiments, the media comprises lactic acid and/or lactates. In some embodiments, the strains may be grown under conditions of anaerobiosis. In some embodiments, the athlete-associated microbial strains disclosed herein are live strains. Without being bound by a theory, it is thought that the ingestion of live athlete-associated microbial strains may enable growth and colonization of the gut tissue in the small and large intestines. The microbial cells may be recovered by centrifugation. Centrifugation may performed, for example at a speed in the range of about 10,000 g to about 15,000 g, such as 12,000 g, for about 15 to about 20 minutes. In some embodiments, the microbial cells may be washed in, for example, an anaerobic phosphate buffer, by resuspension of the cells, agitation, and a further centrifugation step.

In some embodiments, the athlete-associated microbial strains disclosed herein may be processed for commercial purposes using methods, such as fermentation, drying and pulverization. Details of these methods are provided in EP0818529 and WO2001/044440, the contents of each of which are herein incorporated by reference in their entireties for all purposes. In some embodiments, the athlete-associated microbial strains may be concentrated from a medium and dried by spray drying, fluidized bed drying, lyophilization (freeze drying) or other drying processes. In some embodiments, the athlete-associated microbial strains may be mixed with a carrier material such as a carbohydrate such as sucrose, lactose or maltodextrin, a lipid or a protein, for example milk powder during or before the drying.

In some embodiments, the athlete-associated microbial strains disclosed herein may be mixed with a food product directly after fermentation. In some embodiments, a drying process is performed thereafter. Details of these methods are provided in EP0818529 and PCT/EP02/01504, the contents of each of which are herein incorporated by reference in their entireties for all purposes. In some embodiments, the athlete-associated microbial strains disclosed herein may be dried with a food product as described in WO1998/10666, which is incorporated herein by reference in its entirety for all purposes. In some embodiments, the athlete-associated microbial strains disclosed herein may be dried with juices, milk-based products or vegetable milks. Such a product may later be reconstituted with an aqueous liquid. In some embodiments, the athlete-associated microbial strains and/or compositions disclosed herein may be added to a food product, such as a nutritional formula, breakfast cereals, salads, or a slice of bread, prior to consumption.

In some embodiments, the athlete-associated microbial strains disclosed herein may be microencapsulated. Without being bound to a theory, it is thought that microencapsulation formulations and techniques protect the microbes disclosed herein from the digestive actions of the stomach, duodenum, and jejunum of the intestine, and allow administration, delivery or release to the gut or ileum of the subject. Microencapsulated microbes may be co-administered with drugs, foods, nutrients, vitamins, other beneficial substances, prebiotics, and other therapeutic agents such as pH encapsulated glucose, lipids or proteins that release in the distal small intestine at pH values between 7.0 and 8.0. Preferably, at least two coatings are used to cover a tablet or capsule like form comprising the athlete-associated gut microbe, wherein the outside coating is degraded in a pH environment of 5 to 6 and the inside coating is degraded in a pH environment of about 7 thereby releasing the athlete-associated gut microbe in the ileum area and in close proximity to the Peyer's Patches.

An exemplary coating may include one or more of poly(dl-lactide-co-glycolide, chitosan, casein, chitosan (Chi) stabilized with PVA (poly-vinylic alcohol), a lipid, an alginate, carboxymethylethylcellulose (CMEC), cellulose acetate trimellitiate (CAT), hydroxypropylmethyl cellulose phthalate (HPMCP), hydroxypropylmethyl cellulose, ethyl cellulose, color con, food glaze and mixtures of hydroxypropylmethyl cellulose and ethyl cellulose, polyvinyl acetate phthalate (PV AP), cellulose acetate phthalate (CAP), shellac, copolymers of methacrylic acid and ethyl acrylate, and copolymers of methacrylic acid and ethyl acrylate to which a monomer of methylacrylate has been added during polymerization.

The processing steps for producing the athlete-associated microbial strain and/or composition may be used in any order or combination, and any processing step may be omitted or included as determined by a person of skill in the art.

The disclosure further provides a kit including one or more of the athlete-associated microbial strains and/or compositions disclosed herein. In some embodiments, one or more of the athlete-associated microbial strains and/or compositions disclosed herein may be provided within a container. In some embodiments, the kit further comprises one or more devices for measuring units of the athlete-associated microbial strains and/or compositions for administration to the subject, and instructions for the administration.

### Methods of Using the Athlete-Associated Microbial Strains and Compositions Disclosed Herein

The disclosure provides methods of using any one or more of the athlete-associated microbial strains and/or compositions disclosed herein, comprising administering the athlete-associated microbial strains and compositions to the subject. Without being bound to a theory, it is thought that after administration, the microbial strains disclosed herein may adhere to the gut tissue of the subject, such as the wall of the intestine or other gut tissue and multiply, thereby altering the gut microbiome. The disclosure thus provides methods of altering the microbiome of a subject, comprising administering to the subject an effective dose of any one or more of the athlete-associated microbial strains and/or compositions disclosed herein. For instance, in some embodiments, the administration results in increasing the population (or number or abundance) of the one or more athlete-associated gut microbes in the subject. In some embodiments, the administration results in increasing the proportion of the one or more athlete-associated gut microbes in the subject. In some embodiments, the administration results in an increase in the population of the one or more athlete-associated gut microbes in the subject by a value in the range of about 1% to about 1000%, for example about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, or about 1000%, including all subranges and values that lie therebetween.

In some embodiments, the athlete-associated gut microbe, or composition disclosed herein is capable of metabolizing lactic acid and/or lactates. Without being bound to a theory, it is thought that the administration of the one or more athlete-associated gut microbes, or composition disclosed herein disclosed herein capable of metabolizing lactic acid and/or lactates may be associated with a decrease in the level of lactic acid and/or lactates in the subject. Therefore, in some embodiments, the level of lactic acid and/or lactates in the subject is lower after the administration of the one or more athlete-associated gut microbes and/or compositions disclosed herein, as compared to before the administration of the one or more athlete-associated gut microbes and/or compositions disclosed herein. Without being bound by theory, it is thought that lower levels of lactic acid and/or lactates in the blood correlate with higher exercise endurance and/or enhanced recovery from physical exercise.

In some embodiments, the level of lactic acid and/or lactates in the subject is lower by about 1% to about 100%, for example, about 5%, about 10%, about 20%, about 30%, about 40%, about 50%, about 60%, about 70%, about 80%, about 90%, about 100%, including all subranges and values that lie therebetween, after the administration of the one or more athlete-associated gut microbes and/or compositions disclosed herein, as compared to before the administration of the one or more athlete-associated gut microbes and/or compositions disclosed herein. The level of lactic acid and/or lactates may refer the level of lactic acid in the blood, serum, gut, or any other organ, tissue or cell of the subject. In some embodiments, the level of lactic acid and/or lactates is the systemic level of lactic acid and/or lactates.

In some embodiments, the athlete-associated gut microbe is capable of producing propionic acid and/or propionates. Without being bound to a theory, it is thought that the administration of the one or more athlete-associated gut microbes disclosed herein capable of producing propionic acid and/or propionates may be associated with an increase in the level of propionic acid and/or propionates in the subject. In some embodiments, the level of propionic acid or propionates in the subject is higher after the administration of the one or more athlete-associated gut microbes and/or compositions disclosed herein, as compared to before the administration of the one or more athlete-associated gut microbes and/or compositions disclosed herein. Without being bound by theory, it is thought that higher levels of propionic acid and/or propionates in the blood correlate with higher exercise endurance, enhanced recovery from physical exercise, higher heart rate, maximum rate of oxygen consumption, higher energy expenditure, and/or higher lipid oxidation.

In some embodiments, the level of propionic acid or propionates in the subject is higher by about 1% to about 1000%, for example, about 5%, about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, or about 1000%, including all subranges and values that lie therebetween, after the administration of the one or more athlete-associated gut microbes and/or compositions disclosed herein, as compared to before the administration of the one or more athlete-associated gut microbes and/or compositions disclosed herein. The level of propionic acid and/or propionates may refer the level of propionic acid or propionates in the blood, serum, gut, or any other organ, tissue or cell of the subject. In some embodiments, the level of propionic acid or propionates is the systemic level of propionic acid or propionates.

In some embodiments, the athlete-associated gut microbe is capable of producing acetic acid and/or acetates. Without being bound to a theory, it is thought that the administration of the one or more athlete-associated gut microbes disclosed herein capable of producing acetic acid and/or acetates may be associated with an increase in the level of acetic acid and/or acetates in the subject. In some embodiments, the level of acetic acid and/or acetates in the subject is higher after the administration of the one or more athlete-associated gut microbes and/or compositions disclosed herein, as compared to before the administration of the one or more athlete-associated gut microbes and/or compositions disclosed herein. Without being bound by theory, it is thought that higher levels of acetic acid and/or acetates in the blood correlate with higher exercise endurance, enhanced recovery from physical exercise, higher heart rate, maximum rate of oxygen consumption, higher energy expenditure, higher lipid oxidation, and/or enhanced anti-inflammatory effects.

In some embodiments, the level of acetic acid and/or acetates in the subject is higher by about 1% to about 1000%, for example, about 5%, about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, or about 1000%, including all subranges and values that lie therebetween, after the administration of the one or more athlete-associated gut microbes and/or compositions disclosed herein, as compared to before the administration of the one or more athlete-associated gut microbes and/or compositions disclosed herein. The level of acetic acid and/or acetates may refer the level of acetic acid and/or acetates in the blood, serum, gut, or any other organ, tissue or cell of the subject. In some embodiments, the level of acetic acid and/or acetates is the systemic level of acetic acid and/or acetates.

In some embodiments, the administration of the one or more compositions disclosed herein (such as, a composition comprising one or more of the athlete-associated gut microbes disclosed herein, and one or more lactate producing bacteria) is associated with a larger increase in the level of SCFAs, such as acetic acid and/or acetates in the subject, as compared to administration of the one or more of the athlete-associated gut microbes alone. That is, the level of acetic acid and/or acetates in the subject after the administration of the one or more compositions (such as, a composition comprising one or more of the athlete-associated gut microbes disclosed herein, and one or more lactate producing bacteria) disclosed herein is higher, as compared to after the administration of the one or more of the athlete-associated gut microbes alone.

In some embodiments, the level of SCFAs, such as, acetic acid and/or acetates in the subject is higher by about 1% to about 100%, for example, about 5%, about 10%, about 20%, about 30%, about 40%, about 50%, about 60%, about 70%, about 80%, about 90%, about 100%, including all subranges and values that lie therebetween, after the administration of a composition comprising one or more of the athlete-associated gut microbes disclosed herein, and one or more lactate producing bacteria, as compared to after the administration of the one or more of the athlete-associated gut microbes alone.

In some embodiments, the administration of the one or more compositions disclosed herein (such as, a composition comprising one or more of the athlete-associated gut microbes disclosed herein, and one or more lactate producing bacteria) is associated with a decrease in the level of propionic acid and/or propionates in the subject, as compared to administration of the athlete-associated gut microbe alone. That is, the level of propionic acid and/or propionates in the subject after the administration of the one or more compositions (such as, a composition comprising one or more of the athlete-associated gut microbes disclosed herein, and one or more lactate producing bacteria) disclosed herein is lower, as compared to after the administration of the one or more of the athlete-associated gut microbes alone.

In some embodiments, the level of propionic acid and/or propionates in the subject is lower by about 1% to about 100%, for example, about 5%, about 10%, about 20%, about 30%, about 40%, about 50%, about 60%, about 70%, about 80%, about 90%, about 100%, including all subranges and values that lie therebetween, after the administration of a composition comprising one or more of the athlete-associated gut microbes disclosed herein, and one or more lactate producing bacteria, as compared to after the administration of the one or more of the athlete-associated gut microbes alone.

Scheiman et al., Nature Medicine volume 25, pages 1104-1109 (2019) observed that the administration of propionate improved exercise endurance and athletic performance, but did not impact inflammatory cytokines. Acetate, on the other hand, has been shown to improve endurance and have anti-inflammatory properties. Without being bound to any one theory, it is thought that administration of a composition, comprising a Veillonella strain disclosed herein and a lactate producing bacteria to a subject may be beneficial to the subject due to increased production of acetates, which has endurance-improving and anti-inflammatory properties.

In some embodiments, the subject undertakes one or more physical exercise before, during, or after the administration of the one or more athlete-associated gut microbes and/or compositions disclosed herein. In some embodiments, the subject undertakes one or more physical exercise before or after the administration of the one or more athlete-associated gut microbes and/or compositions disclosed herein; wherein the time period between the administration of the one or more athlete-associated gut microbe and undertaking of physical exercise is not limited, and may be, for example, in the range of about 1 minute to about several months, for example, 5 min, 30 min, 1 hour, 6 hours, 12 hours, 24 hours, 2 days, 3 days, 4 days, 5 days, 6 days, 1 week, 2 weeks, 3 weeks, 1 month, 1 year, including all subranges and values that lie therebetween.

In some embodiments, the level of lactic acid and/or lactates in the subject, who is administered one or more athlete-associated microbial strains and/or compositions disclosed herein, is lower after the physical exercise, as compared to a control subject. As used herein, a “control subject” is a subject who undertakes the same physical exercise as the subject, and is otherwise similar to the subject based on physiological factors, but is not administered the one or more athlete-associated gut microbes and/or compositions disclosed herein. In some embodiments, the level of lactic acid and/or lactates in the subject after physical exercise is lower by about 1% to about 100%, for example, about 5%, about 10%, about 20%, about 30%, about 40%, about 50%, about 60%, about 70%, about 80%, about 90%, about 100%, including all subranges and values that lie therebetween, as compared to the level of lactic acid and/or lactates in the control subject after physical exercise.

In some embodiments, the level of propionic acid, propionates, acetic acid, and/or acetates in the subject, who is administered one or more athlete-associated microbial strains and/or compositions disclosed herein, is higher after the physical exercise, as compared to the control subject. In some embodiments, the level of propionic acid, propionates, acetic acid, and/or acetates in the subject after physical exercise is higher by about 1% to about 100%, for example, about 5%, about 10%, about 20%, about 30%, about 40%, about 50%, about 60%, about 70%, about 80%, about 90%, about 100%, including all subranges and values that lie therebetween, as compared to the level of propionic acid, propionates acetic acid, and/or acetates in the control subject after physical exercise.

In some embodiments, the level of SCFAs, such as, acetic acid and/or acetates in the subject, who is administered a composition disclosed herein, comprising one or more of the athlete-associated gut microbes disclosed herein, and one or more lactate producing bacteria, is higher after the physical exercise, as compared to a subject, who is administered one or more of the athlete-associated gut microbes alone. In some embodiments, the level of acetic acid and/or acetates in the subject, who is administered a composition disclosed herein, comprising one or more of the athlete-associated gut microbes disclosed herein, and one or more lactate producing bacteria, is higher after physical exercise by about 1% to about 100%, for example, about 5%, about 10%, about 20%, about 30%, about 40%, about 50%, about 60%, about 70%, about 80%, about 90%, about 100%, including all subranges and values that lie therebetween, as compared to a subject, who is administered one or more of the athlete-associated gut microbes alone.

In some embodiments, the administration of the one or more athlete-associated gut microbes and/or compositions disclosed herein to the subject is associated with, promotes or causes enhanced exercise endurance, improved athletic performance, reduced inflammation, enhanced recovery from physical exercise, a decrease in fatigue, or any combination thereof in the subject. Without being bound by a theory, it is thought that a decrease in the levels of lactic acid and/or lactates, for example in the blood of the subject, and/or an increase in the levels of propionic acid, propionates, acetic acid, and/or acetates for example in the blood of the subject may be associated with, promote or cause enhanced exercise endurance, improved athletic performance, reduced inflammation, enhanced recovery from physical exercise, a decrease in fatigue, or any combination thereof in the subject.

Thus, the disclosure provides methods of enhancing exercise endurance of a subject, comprising administering to the subject any one or more of the athlete-associated gut microbes and/or compositions disclosed herein. The disclosure further provides methods of improving the athletic performance of a subject, comprising administering to the subject any one or more of the athlete-associated gut microbes and/or compositions disclosed herein.

In some embodiments, the method comprises an improvement of one or more objective factors related to a physical exercise for the subject, who is administered one or more athlete-associated microbial strains and/or compositions disclosed herein. In some embodiments, the physical exercise is running. Therefore, in some embodiments, the method comprises an improvement of one or more objective factors related to running, such as, for example, the maximum time the subject is able to run until exhaustion point (also called, “maximum run time”), the maximum distance run until exhaustion point (also called, “maximum run distance”), the distance run in a particular time period, and the time taken to run a particular distance. In some embodiments, the maximum run time of the subject who is administered the one or more athlete-associated gut microbes and/or compositions disclosed herein is increased by about 1% to about 100%, for example, about 5%, about 10%, about 20%, about 30%, about 40%, about 50%, about 60%, about 70%, about 80%, about 90%, about 100%, including all subranges and values that lie therebetween, as compared to the control subject. As used herein, a control subject undertakes the same physical exercise as the subject (that is, running), and is otherwise similar to the subject based on physiological factors, but is not administered the one or more athlete-associated gut microbes and/or compositions disclosed herein. In some embodiments, the maximum run distance of the subject who is administered the one or more athlete-associated gut microbes and/or compositions disclosed herein is increased by about 1% to about 100%, for example, about 5%, about 10%, about 20%, about 30%, about 40%, about 50%, about 60%, about 70%, about 80%, about 90%, about 100%, including all subranges and values that lie therebetween, as compared to the control subject. In some embodiments, the distance run in a particular time period of the subject who is administered the one or more athlete-associated gut microbes and/or compositions disclosed herein is increased by about 1% to about 100%, for example, about 5%, about 10%, about 20%, about 30%, about 40%, about 50%, about 60%, about 70%, about 80%, about 90%, about 100%, including all subranges and values that lie therebetween, as compared to the control subject. In some embodiments, the time taken to run a particular distance of the subject who is administered the one or more athlete-associated gut microbes and/or compositions disclosed herein is decreased by about 1% to about 95%, for example, about 5%, about 10%, about 20%, about 30%, about 40%, about 50%, about 60%, about 70%, about 80%, or about 90%, including all subranges and values that lie therebetween, as compared to the control subject.

In some embodiments, the methods comprise a reduction in the level of at least one inflammatory cytokine in the subject after the administration of the one or more athlete-associate microbes and/or compositions disclosed herein, as compared to before the administration of the one or more athlete-associate microbes and/or compositions disclosed herein. In some embodiments, the level of the at least one inflammatory cytokine in the subject is lower by about 1% to about 100%, for example, about 5%, about 10%, about 20%, about 30%, about 40%, about 50%, about 60%, about 70%, about 80%, about 90%, about 100%, including all subranges and values that lie therebetween, after the administration of the one or more athlete-associated gut microbes and/or compositions disclosed herein, as compared to before the administration of the one or more athlete-associated gut microbes and/or compositions disclosed herein. Without being bound by a theory, it is thought that lower levels of inflammatory cytokines is correlated with reduced inflammation and/or enhanced recovery from physical exercise. Therefore, the disclosure provides methods of reducing inflammation in a subject in need thereof, comprising administering to the subject any one or more of the athlete-associated gut microbes and/or compositions disclosed herein. The disclosure further provides methods of enhancing recovery from physical exercise in a subject in need thereof, comprising administering to the subject any one or more of the athlete-associated gut microbes and/or compositions disclosed herein.

In some embodiments, the level of the at least one inflammatory cytokine after physical exercise in the subject, who is administered one or more microbial strains and/or composition disclosed herein, is lower by about 1% to about 100%, for example, about 5%, about 10%, about 20%, about 30%, about 40%, about 50%, about 60%, about 70%, about 80%, about 90%, about 100%, including all subranges and values that lie therebetween, as compared to the level of the at least one inflammatory cytokine in the control subject after physical exercise.

The disclosure further provides methods of increasing muscle mass and/or muscular strength of a subject in need thereof, comprising administering to the subject an effective dose of any one or more of the athlete-associated gut microbes and/or compositions disclosed herein. The disclosure further provides methods of preventing the loss of muscle mass in a subject in need thereof, comprising administering to the subject an effective dose of any one or more of the athlete-associated gut microbes and/or compositions disclosed herein. In some embodiments, muscle loss may be age-related muscle loss (sarcopenia), cachexia, or muscle disuse atrophy due to for instance, hospitalization, immobilization, or injury. In some embodiments, muscle loss may be due to intake of calorie-deficient diets.

The effective dosage of the one or more of the athlete-associated microbial strains disclosed herein is not limited, and may be determined by the subject, either alone, or in consultation with a person skilled in the art, physician or athletic trainer. In some embodiments, the dosage enables the successful colonization of the gut tissue. In some embodiments, the dosage may be in range of about 10⁴ colony forming units (CFU) to 10¹⁶ CFU, for example, about 10⁵, about 10⁶, about 10⁷, about 10⁸, about 10⁹, about 10¹⁰, about 10¹¹, about 10¹², about 10¹³, about 10¹⁴, or about 10¹⁵, including all subranges and values that lie therebetween. In some embodiments, the dosage is in the range of about 10⁹ to about 10¹¹ CFU. In some embodiments, the dosage in the range of about 5×10⁹ CFU to about 10¹⁰ CFU. In some embodiments, the dosage in the range of about 5×10⁹ CFU to about 15×10⁹ CFU.

The frequency of dosage administered is not limited, and may be determined by a person skilled in the art, either alone, or in consultation with a physician or athletic trainer. In some embodiments, the one or more of the athlete-associated microbial strains may be administered to the subject once a day or more than once a day. In some embodiments, the one or more of the athlete-associated microbial strains may be administered to the subject twice, thrice, four, five, six, 7, 8, 9 or 10 times a day. In some embodiments, the one or more of the athlete-associated microbial strains may be administered to the subject weekly or a few times a week. In some embodiments, the one or more of the athlete-associated microbial strains may be administered to the subject every day, every alternate, every third day, every fourth day, every fifth day, or every sixth day. In some embodiments, the one or more of the athlete-associated microbial strains may be administered to the subject weekly, bi-weekly or every three weeks. In some embodiments, the one or more of the athlete-associated microbial strains may be administered to the subject every 1, 2, 3, 4, 5, 6, 7, 8, 9, 10 or 11 months. In some embodiments, the one or more of the athlete-associated microbial strains may be administered to the subject every year. In some embodiments, the one or more of the athlete-associated microbial strains may be administered to the subject every 1, 2, 3, 4, 5, 10, 15, or 20 years. In some embodiments, the one or more of the athlete-associated microbial strains may be administered to the subject before, during or after performing a physical exercise.

Administration of the one or more of the athlete-associated microbial strains and/or compositions disclosed herein to the subject may be any route that is determined to be effective by a person skilled in the art, a physician or an athletic trainer. In some embodiments, the administration is via oral, enteric, gastrointestinal, rectal, or parenteral route.

The disclosure provides methods of generating model animals, comprising administering or transplanting any one or more of athlete-associated gut microbes and/or compositions disclosed herein into the animal. In some embodiments, the transplantation is done to the gut tissue of the animal. In some embodiments, the model animal is a mouse, for example a germ-free mouse. In some embodiments, the methods further comprise screening the model animals transplanted with any one or more of athlete-associated gut microbes and/or compositions to determine their physiological and physical characteristics.

The disclosure also provides methods of improving the athletic performance of an animal, comprising administering any one or more athlete-associated gut microbes and/or compositions disclosed herein to the animal. In some embodiments, the administration is via oral, enteric, gastrointestinal, or rectal route. In some embodiments, the animal is a horse.

The terms and expressions that have been employed are used as terms of description and not of limitation, and there is no intent in the use of such terms and expressions to exclude any equivalent of the features reported and described or portions thereof, but it is recognized that various modifications are possible within the scope of the invention as claimed. Thus, it will be understood that although the present disclosure includes preferred embodiments and optional features, modification and variation of the concepts herein disclosed may be resorted to by those skilled in the art, and that such modifications and variations are considered to be within the scope of the appended claims.

### Budapest Treaty on the International Recognition of the Deposit of Microorganisms for the Purpose of Patent Procedures

Microorganisms described in this Application were deposited with the American Type Culture Collection (ATCC®), located at 10801 University Blvd., Manassas, Va. 20110, USA. The deposits were made under the terms of the Budapest Treaty on the International Recognition of the Deposit of Microorganisms for the Purposes of Patent Procedure. The ATCC accession numbers for the aforementioned Budapest Treaty deposits are provided herein.

A representative sample of the Veillonella dispar isolate described herein has been deposited on Oct. 15, 2020, under ATCC accession number PTA-126861.

A representative sample of the Veillonella parvula isolate described herein has been deposited on Oct. 15, 2020, under ATCC accession number PTA-126859.

A representative sample of the Veillonella atypica isolate described herein has been deposited on Oct. 15, 2020, under ATCC accession number PTA-126860.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

The following examples are set forth as being representative of the present disclosure. These examples are not to be construed as limiting the scope of the present disclosure as these and other equivalent embodiments will be apparent in view of the present disclosure, figures and accompanying claims.

### EXAMPLES

### Example 1— Identification and Isolation of New, Specific Athlete-Associated Gut Microbes

To identify athlete-associated gut microbes, athletes were recruited and requested to provide stool samples before and after strenuous physical activity (exercise, training, performance, competition, etc.). For the collection of stool, athletes were provided with a 15 ml falcon tube with a 1 ml pipette tip inserted inside. Participants were instructed to dip the pipette tips into soiled toilet tissue, then place them back into the tubes and label the tubes with the date and time of collection. Samples were kept at 4° C. for short-term storage until sample pickup, at which point they were immediately placed onto dry ice, then transferred to a −80° C. freezer for long-term storage. Stool samples were thawed on ice and resuspended in 2-5 ml of PBS, 250 μl of which was used for DNA extraction using the Mo Bio PowerSoil high-throughput DNA extraction kit, following the manufacturer's protocol. For genus/OTU level identification, 16S ribosomal DNA (rDNA) analysis was conducted utilizing 1-5 μl of purified DNA for PCR amplification of the V4 variable region. 16S rDNA amplicon sequencing was followed by bioinformatic analysis, utilizing either open access software tools such as DADA2 and Qiime or custom scripts. For species/strain level identification, and gene functional annotation, whole genome shotgun analysis was conducted using 1 ng of purified DNA for Illumina's Nextera XI Tagmentation kit. Shotgun sequencing was followed by bioinformatic analysis, utilizing open access software tools such as Metaphlan, Humann, megahit, and prokka, or custom analysis scripts

Through analysis of the sequencing data, the following strains were identified as being significantly more prevalent among the elite athletes, as compared to non-athlete controls, as well as enriched after strenuous physical activity: a Veillonella dispar strain comprising a 16S rRNA gene comprising a nucleic acid sequence of SEQ ID NO: 1; a Veillonella parvula strain comprising a 16S rRNA gene comprising a nucleic acid of SEQ ID NO: 11; and a Veillonella atypica strain comprising a 16S rRNA gene comprising a nucleic acid of SEQ ID NO: 21. Veillonella species (V. dispar, V. parvula and V. atypica) were isolated and purified from several athletes by plating stool, resuspended in PBS, onto lactate agar petri dishes (per liter: 5 g bacto yeast extract, 0.75 g sodium thioglycolate, 25 ml basic fuchsin and 21 ml 60% sodium lactate, 15 g bacto agar, 7.5 ug/ml vancomycin. (pH 7.5)) and grown under anaerobic conditions. Individuals colonies were selected and identified initially using MALDI-TOF mass spectrometry, followed by 1.6S rRNA and whole genome shotgun sequencing methodologies as described above. Veillonella species were then propagated and maintained in three different media compositions: (1) BHI supplemented with lactate (10 ml of 60% sodium lactate per liter); (2) MRS broth supplemented with lactate (10 ml of 60% sodium lactate per liter); and (3) lactate medium. Veillonella species were inoculated into each medium and grown under anaerobic conditions. These strains were deposited with the American Type Culture Collection (ATCC®).

### Example 2— Characterization of New Athlete-Associated Gut Microbe

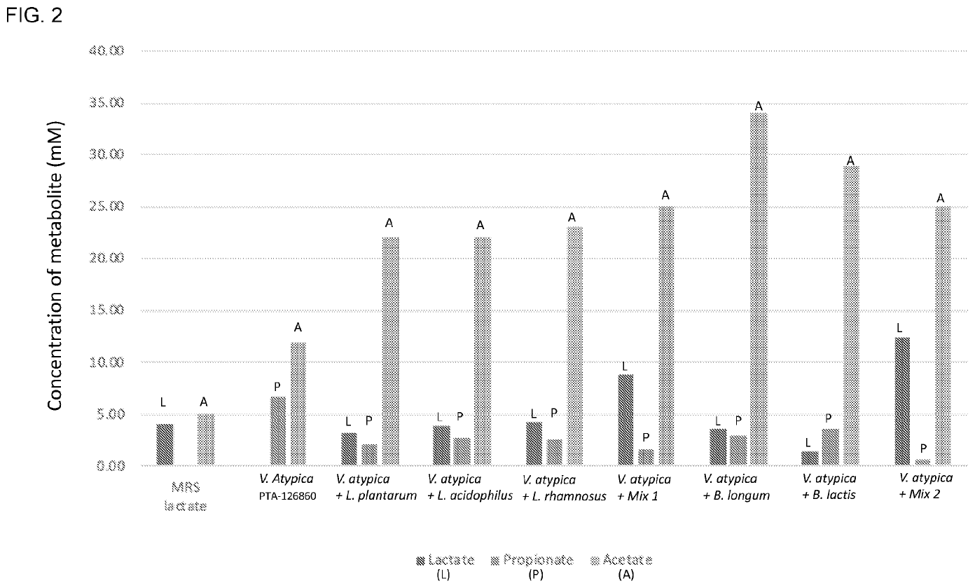

The ability of Veillonella atypica PTA-126860 to convert lactate to propionate and acetate was observed by Mass Spectrometry. Veillonella atypica PTA-126860 was cultured in MRS lactate medium, which contains sodium lactate, a nutrient source for Veillonella strains, for 48 hours at 37° C. anaerobically. MRS lactate medium alone was used as a negative or baseline control. FIG. 2 shows that Veillonella atypica PTA-126860 produces both acetate and propionate from lactate, as compared to the baseline control. This result verifies that the newly identified Veillonella atypica PTA-126860 strain is capable of producing performance enhancing molecules, such as acetate and propionate. Therefore, this supports that the administration of Veillonella atypica PTA-126860 to a subject might improve athletic performance in the subject by increasing the levels of performance-enhancing molecules, such as acetate and propionate, in the subject.

### Example 3— Characterization of Compositions Comprising Athlete-Associated Gut Microbes and Lactate Producing Bacteria

Compositions comprising Veillonella atypica PTA-126860 and any one of the following lactate-producing microbes were generated: FB00015 Lactobacillus plantarum, FB00012 Lactobacillus acidophilus, FB00047 Lactobacillus rhamnosus, FB00032 Bifidobacterium longum, and FB00034 Bifidobacterium lactis. In addition, compositions comprising Veillonella atypica PTA-126860 and Mix 1 (comprising FB00015 L. plantarum, FB00012 L. acidophilus, FB00047 L. rhamnosus) or Mix 2 (comprising FB00015 L. plantarum, FB00012 L. acidophilus, FB00047 L. rhamnosus, FB00032 B. longum and FB00034 B. lactis) were also generated. Table 1 lists the 16S rRNA sequences of the additional strains in these compositions. For each of the compositions, equal amounts of each of the constituent strains were used. In other words, a ratio of 1:1 was used for a composition with 2 microbes, a ratio of 1:1:1:1 was used for a composition with 4 microbes and so on. The compositions were cultured in MRS lactate medium for 48 hours at 37° C. anaerobically. MRS lactate medium alone was used as a negative or baseline control. The ability of the compositions to convert lactate to propionate and acetate was observed by Mass Spectrometry.

TABLE 1

| Strain | Sequence of 16S rRNA |
|---|---|
| FB00015 L. plantarum | SEQ ID NO: 61 |
| FB00012 L. acidophilus | SEQ ID NO: 62 |
| FB00047 L. rhamnosus | SEQ ID NO: 63 |
| FB00032 B. longum | SEQ ID NO: 64 |
| FB00034 B. lactis | SEQ ID NO: 65 |

Surprisingly, there is a striking increase in the amount of acetate produced by each of the tested compositions, as compared to Veillonella atypica PTA-126860 alone (FIG. 2). For instance, the composition comprising Veillonella atypica PTA-126860 and Bifidobacterium longum produces >3 fold more acetate than Veillonella atypica PTA-126860 alone. Also, each of the compositions tested produced less propionate than Veillonella atypica PTA-126860 alone (FIG. 2). Overall, these results demonstrate that compositions comprising an athlete-associated gut microbe, such as, Veillonella atypica PTA-126860, and a lactate producing bacteria produce more acetate, as compared to the Veillonella atypica PTA-126860 strain on its own.

### Example 4— Characterization of New Athlete-Associated Gut Microbes in Model Organisms and Humans

Human feasibility studies will be performed to determine Veillonella species GI transit time and viability, as well as lactate metabolism functionality via stool metagenomic and metabolomic analysis. This will be followed by human performance testing using double blind placebo controlled cross over studies to determine the effects of Veillonella species consumption on VO2 max, exercise capacity, endurance, inflammation, recovery, and strength applications. Veillonella species will be tested in different doses as well as in combination with potentially synergistic compounds (vitamins, electrolytes, prebiotics, lactate, prebiotics, etc.). Pre-clinical studies will be performed in mice to explore additional functional benefits of Veillonella species, including: 1) Strength and protein metabolism/absorption 2) Neurological and gut-brain access applications (sleep, stress, anxiety social interactions, cognition) 3) Inflammation (gut and distal location in the body) and 4) Nutrition and digestion.

### (1) Human Performance Testing of Veillonella Strains

A cohort of human subjects (for example, —50 subjects) will be administered about 5-10 billion CFU of either one, or any combination of, the following bacteria: Veillonella atypica having deposit accession number PTA-126860, Veillonella dispar having deposit accession number PTA-126861, and Veillonella parvula having deposit accession number PTA-126859.

Health indicators (such as, the diversity of the microbiome, the blood levels of lactates, acetates and propionates, exercise endurance, athletic performance, levels of inflammatory cytokines and other markers of inflammation, ability to recover from physical exercise, muscle mass or muscular strength, sleep quality, sleep length, and mental health indicators) will be measured for these subjects before and after administration of the Veillonella strains for a period of time (e.g., 2 weeks). A comparison of these health indicators will also be done with human subjects who were administered a placebo over this time period.

After administration of the aforementioned Veillonella strains, human subjects will show any one of: an altered microbiome, improved diversity of microbiome, reduced blood levels of lactates, increased blood levels of propionates and/or acetates, enhanced exercise endurance, improved athletic performance, reduced inflammation, reduced levels of inflammatory cytokines and/or other markers of inflammation, enhanced ability to recover from physical exercise, increased muscle mass or muscular strength, decreased muscle loss, better quality of sleep, longer sleep, and/or improved mental health indicators, and/or any combination thereof, as compared to before administration, or human subjects on placebo.

(2) Human Performance Testing of Veillonella Strains in Combination with Lactate Producing Bacteria

A cohort of human subjects (for example, —50 subjects) will be administered about 5-10 billion CFU of: (a) Veillonella atypica having deposit accession number PTA-126860, Veillonella dispar having deposit accession number PTA-126861, and/or Veillonella parvula having deposit accession number PTA-126859; and (b) Lactobacillus plantarum comprising a 16S rRNA nucleic acid sequence that is at least 97% identical to SEQ ID NO: 61; Lactobacillus acidophilus comprising a 16S rRNA nucleic acid sequence that is at least 97% identical to SEQ ID NO: 62; Lactobacillus rhamnosus comprising a 16S rRNA nucleic acid sequence that is at least 97% identical to SEQ ID NO: 63; Lactobacillus paracasei comprising a 16S rRNA nucleic acid sequence that is at least 97% identical to SEQ ID NO: 66; Bifidobacterium longum comprising a 16S rRNA nucleic acid sequence that is at least 97% identical to SEQ ID NO: 64; and/or Bifidobacterium lactis comprising a 16S rRNA nucleic acid sequence that is at least 97% identical to SEQ ID NO: 65.

For example, one cohort of human subjects will be administered Veillonella atypica having deposit accession number PTA-126860, in combination with aforementioned Lactobacillus and Bifidobacterium strains. As another example, another cohort of human subjects will be administered Veillonella atypica having deposit accession number PTA-126860, in combination with just the aforementioned Lactobacillus strains.

Health indicators (such as, the diversity of the microbiome, the blood levels of lactates, acetates and propionates, exercise endurance, athletic performance, levels of inflammatory cytokines and other markers of inflammation, ability to recover from physical exercise, muscle mass or muscular strength, sleep quality, sleep length, and mental health indicators) will be measured for these subjects before and after administration of the microbes or compositions described herein for a period of time (e.g., 2 weeks). A comparison of these health indicators will also be done with human subjects who were administered a placebo, and with human subjects who were administered just Veillonella strains over this time period.

After administration of the aforementioned combinations, human subjects will show any one of: an altered microbiome, improved diversity of microbiome, reduced blood levels of lactates, increased blood levels of propionates and/or acetates, enhanced exercise endurance, improved athletic performance, reduced inflammation, reduced levels of inflammatory cytokines and/or other markers of inflammation, enhanced ability to recover from physical exercise, increased muscle mass or muscular strength, decreased muscle loss, better quality of sleep, longer sleep, and/or improved mental health indicators, and/or any combination thereof, as compared to before administration, or human subjects on placebo, or human subjects who were administered just Veillonella strains.

### Example 5— Characterization of Metabolic Pathways in Athlete-Associated Gut Microbes

Dozens of additional athlete-associated Veillonella species and strains (variations of V. dispar, V. parvula, V. atypical) will be identified and isolated for comprehensive metagenomic functional annotation/analysis—looking to identify genes and metabolic pathways contributing to beneficial growth, SCFA synthesis, energy and protein metabolism, immunological and digestive health, as well as other beneficial physiological processes. Annotated genes will be used to develop probiotic, nutritional, fitness, health and wellness, as well as synthetic biology markers and tools. Veillonella species will be grown in various culture media (BHI, MRS, lactate, etc.) to assess beneficial functions in terms of metabolite, neurotransmitter, lipid, and proteomic production as well as metabolic, enzymatic, fermentation, and growth capabilities. Veillonella species will be grown in conjunction with additional athlete associated gut microbes, substrates, nutrients, and prebiotics to look for synergistic affects. Mass spectrometry (MS) will be performed on the cultured media to determine the identity of the compounds that are produced by the athlete-associated gut microbes. It is expected that the athlete-associated gut microbes identified here will metabolize lactates in the media to produce products, such as acetate and propionates, which can be identified by MS.

### Further Numbered Embodiments

Embodiment 1. An isolated and purified Veillonella dispar, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 98.6% sequence identity to SEQ ID NO: 2; (ii) VR2 comprises a nucleic acid sequence with at least 98.6% sequence identity to SEQ ID NO: 3; (iii) VR3 comprises a nucleic acid sequence with at least 98.6% sequence identity to SEQ ID NO: 4; (iv) VR4 comprises a nucleic acid sequence with at least 98.6% sequence identity to SEQ ID NO: 5; (v) VR5 comprises a nucleic acid sequence with at least 98.6% sequence identity to SEQ ID NO: 6; (vi) VR6 comprises a nucleic acid sequence with at least 98.6% sequence identity to SEQ ID NO: 7; (vii) VR7 comprises a nucleic acid sequence with at least 98.6% sequence identity to SEQ ID NO: 8; (viii) VR8 comprises a nucleic acid sequence with at least 98.6% sequence identity to SEQ ID NO: 9; and (ix) VR9 comprises a nucleic acid sequence with at least 98.6% sequence identity to SEQ ID NO: 10.
Embodiment 2. The Veillonella dispar of embodiment 1, wherein the nucleic acid sequence comprises at least one constant region (CR) selected from the group consisting of: CR1, CR2, CR3, CR4, CR5, CR6, CR7, CR8, CR9, CR10, and any combination thereof, wherein (i) CR1 comprises a nucleic acid sequence with at least 80% sequence identity to SEQ ID NO: 31; (ii) CR2 comprises a nucleic acid sequence with at least 80% sequence identity to SEQ ID NO: 32; (iii) CR3 comprises a nucleic acid sequence with at least 80% sequence identity to SEQ ID NO: 33; (iv) CR4 comprises a nucleic acid sequence with at least 80% sequence identity to SEQ ID NO:34; (v) CR5 comprises a nucleic acid sequence with at least 80% sequence identity to SEQ ID NO: 35; (vi) CR6 comprises a nucleic acid sequence with at least 80% sequence identity to SEQ ID NO: 36; (vii) CR7 comprises a nucleic acid sequence with at least 80% sequence identity to SEQ ID NO: 37; (viii) CR8 comprises a nucleic acid sequence with at least 80% sequence identity to SEQ ID NO: 38; (ix) CR9 comprises a nucleic acid sequence with at least 80% sequence identity to SEQ ID NO: 39; and (x) CR10 comprises a nucleic acid sequence with at least 80% sequence identity to SEQ ID NO: 40.
Embodiment 3. The Veillonella dispar of embodiment 1 or embodiment 2, wherein the nucleic acid sequence comprises at least 98.6% sequence identity to SEQ ID NO: 1.
Embodiment 4. An isolated and purified Veillonella dispar, comprising: a 16S rRNA gene comprising a nucleic acid sequence with at least 98.6% sequence identity to SEQ ID NO: 1.
Embodiment 5. An isolated and purified Veillonella parvula, comprising: a 16S rRNA gene comprising a nucleic acid sequence comprising at least one variable region (VR) selected from the group consisting of VR1, VR2, VR3, VR4, VR5, VR6, VR7, VR8, VR9, and any combination thereof, wherein (i) VR1 comprises a nucleic acid sequence with at least 98.6% sequence identity to SEQ ID NO: 12; (ii) VR2 comprises a nucleic acid sequence with at least 98.6% sequence identity to SEQ ID NO: 13; (iii) VR3 comprises a nucleic acid sequence with at least 98.6% sequence identity to SEQ ID NO: 14; (iv) VR4 comprises a nucleic acid sequence with at least 98.6% sequence identity to SEQ ID NO: 15; (v) VR5 comprises a nucleic acid sequence with at least 98.6% sequence identity to SEQ ID NO: 16; (vi) VR6 comprises a nucleic acid sequence with at least 98.6% sequence identity to SEQ ID NO: 17; (vii) VR7 comprises a nucleic acid sequence with at least 98.6% sequence identity to SEQ ID NO: 18; (viii) VR8 comprises a nucleic acid sequence with at least 98.6% sequence identity to SEQ ID NO: 19; and (ix) VR9 comprises a nucleic acid sequence with at least 98.6% sequence identity to SEQ ID NO: 20.
Embodiment 6. The Veillonella parvula of embodiment 5, wherein the nucleic acid sequence comprises at least one constant region (CR) selected from the group consisting of: CR1, CR2, CR3, CR4, CR5, CR6, CR7, CR8, CR9, CR10, and any combination thereof, wherein (i) CR1 comprises a nucleic acid sequence with at least 80% sequence identity to SEQ ID NO: 41; (ii) CR2 comprises a nucleic acid sequence with at least 80% sequence identity to SEQ ID NO: 42; (iii) CR3 comprises a nucleic acid sequence with at least 80% sequence identity to SEQ ID NO: 43; (iv) CR4 comprises a nucleic acid sequence with at least 80% sequence identity to SEQ ID NO: 44; (v) CR5 comprises a nucleic acid sequence with at least 80% sequence identity to SEQ ID NO: 45; (vi) CR6 comprises a nucleic acid sequence with at least 80% sequence identity to SEQ ID NO: 46; (vii) CR7 comprises a nucleic acid sequence with at least 80% sequence identity to SEQ ID NO: 47; (viii) CR8 comprises a nucleic acid sequence with at least 80% sequence identity to SEQ ID NO: 48; (ix) CR9 comprises a nucleic acid sequence with at least 80% sequence identity to SEQ ID NO: 49; and (x) CR10 comprises a nucleic acid sequence with at least 80% sequence identity to SEQ ID NO: 50.
Embodiment 7. The Veillonella parvula of embodiment 5 or embodiment 6, wherein the nucleic acid sequence comprises at least 98.6% sequence identity to SEQ ID NO: 11.
Embodiment 8. An isolated and purified Veillonella parvula, comprising: a 16S rRNA gene comprising a nucleic acid sequence with at least 98.6% sequence identity to SEQ ID NO: 11.
Embodiment 9. An isolated and purified Veillonella atypica, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 98.6% sequence identity to SEQ ID NO: 22; (ii) VR2 comprises a nucleic acid sequence with at least 98.6% sequence identity to SEQ ID NO: 23; (iii) VR3 comprises a nucleic acid sequence with at least 98.6% sequence identity to SEQ ID NO: 24; (iv) VR4 comprises a nucleic acid sequence with at least 98.6% sequence identity to SEQ ID NO: 25; (v) VR5 comprises a nucleic acid sequence with at least 98.6% sequence identity to SEQ ID NO: 26; (vi) VR6 comprises a nucleic acid sequence with at least 98.6% sequence identity to SEQ ID NO: 27; (vii) VR7 comprises a nucleic acid sequence with at least 98.6% sequence identity to SEQ ID NO: 28; (viii) VR8 comprises a nucleic acid sequence with at least 98.6% sequence identity to SEQ ID NO: 29; and (ix) VR9 comprises a nucleic acid sequence with at least 98.6% sequence identity to SEQ ID NO: 30.
Embodiment 10. The Veillonella atypica of embodiment 9, wherein the nucleic acid sequence comprises at least one constant region (CR) selected from the group consisting of CR1, CR2, CR3, CR4, CR5, CR6, CR7, CR8, CR9, CR10 and any combination thereof, wherein (i) CR1 comprises a nucleic acid sequence with at least 80% sequence identity to SEQ ID NO: 51; (ii) CR2 comprises a nucleic acid sequence with at least 80% sequence identity to SEQ ID NO: 52; (iii) CR3 comprises a nucleic acid sequence with at least 80% sequence identity to SEQ ID NO: 53; (iv) CR4 comprises a nucleic acid sequence with at least 80% sequence identity to SEQ ID NO: 54; (v) CR5 comprises a nucleic acid sequence with at least 80% sequence identity to SEQ ID NO: 55; (vi) CR6 comprises a nucleic acid sequence with at least 80% sequence identity to SEQ ID NO: 56; (vii) CR7 comprises a nucleic acid sequence with at least 80% sequence identity to SEQ ID NO: 57; (viii) CR8 comprises a nucleic acid sequence with at least 80% sequence identity to SEQ ID NO: 58; (ix) CR9 comprises a nucleic acid sequence with at least 80% sequence identity to SEQ ID NO: 59; and (x) CR10 comprises a nucleic acid sequence with at least 80% sequence identity to SEQ ID NO: 60.
Embodiment 11. The Veillonella atypica of embodiment 9 or embodiment 10, wherein the nucleic acid sequence comprises at least 98.6% sequence identity to SEQ ID NO: 21.
Embodiment 12. An isolated and purified Veillonella atypica, comprising: a 16S rRNA gene comprising a nucleic acid sequence with at least 98.6% sequence identity to SEQ ID NO: 21.
Embodiment 13. An isolated and purified Veillonella dispar having deposit accession number PTA-126861, or a strain having all of the identifying characteristics of Veillonella dispar PTA-126861, or a mutant thereof.
Embodiment 14. An isolated and purified Veillonella parvula having deposit accession number PTA-126859, or a strain having all of the identifying characteristics of Veillonella parvula PTA-126859, or a mutant thereof.
Embodiment 15. An isolated and purified Veillonella atypica having deposit accession number PTA-126860, or a strain having all of the identifying characteristics of Veillonella atypica PTA-126860, or a mutant thereof.
Embodiment 16. A composition comprising the Veillonella dispar of any one of embodiments 1-4 and 13.
Embodiment 17. A composition comprising the Veillonella parvula of any one of embodiments 5-8 and 14.
Embodiment 18. A composition comprising the Veillonella atypica of any one of embodiments 9-12 and 15.
Embodiment 19. A composition comprising the Veillonella sp. strain of any one of embodiments 1-15.
Embodiment 20. The composition of embodiment 19, wherein the composition comprises one or more lactate producing bacteria.
Embodiment 21. The compositions of embodiment 20, wherein the lactate-producing bacteria belongs to the genus Lactobacillus or Bifidobacterium.
Embodiment 22. The composition of embodiment 21, wherein the lactate-producing bacteria is Lactobacillus plantarum, Lactobacillus acidophilus, Lactobacillus rhamnosus, Bifidobacterium longum, Bifidobacterium lactis, or any combination thereof.
Embodiment 23. The composition of any one of embodiments 20-22, wherein the composition produces more acetate than the Veillonella sp. strain.
Embodiment 24. The composition of any one of embodiments 16-23, wherein the composition is a food composition, a beverage composition or a dietary supplement composition.
Embodiment 25. A composition, comprising: a Veillonella sp. strain of any one of embodiments 1-15, and any one or more of the following strains:
(a) an isolated and purified Lactobacillus plantarum comprising a 16S rRNA nucleic acid sequence that is at least 97% identical to SEQ ID NO: 61;
(b) an isolated and purified Lactobacillus acidophilus comprising a 16S rRNA nucleic acid sequence that is at least 97% identical to SEQ ID NO: 62;
(c) an isolated and purified Lactobacillus rhamnosus comprising a 16S rRNA nucleic acid sequence that is at least 97% identical to SEQ ID NO: 63;
(d) an isolated and purified Bifidobacterium longum comprising a 16S rRNA nucleic acid sequence that is at least 97% identical to SEQ ID NO: 64;
(e) an isolated and purified Bifidobacterium lactis comprising a 16S rRNA nucleic acid sequence that is at least 97% identical to SEQ ID NO: 65; and an isolated and purified Lactobacillus paracasei comprising a 16S rRNA nucleic acid sequence that is at least 97% identical to SEQ ID NO: 66.
Embodiment 26. A composition, comprising: a Veillonella sp. strain of any one of embodiments 1-15, and any one or more of the following strains:
(a) an isolated and purified Lactobacillus plantarum comprising a 16S rRNA nucleic acid sequence that is at least 97% identical to SEQ ID NO: 61;
(b) an isolated and purified Lactobacillus acidophilus comprising a 16S rRNA nucleic acid sequence that is at least 97% identical to SEQ ID NO: 62;
(c) an isolated and purified Lactobacillus rhamnosus comprising a 16S rRNA nucleic acid sequence that is at least 97% identical to SEQ ID NO: 63; and
(d) an isolated and purified Lactobacillus paracasei comprising a 16S rRNA nucleic acid sequence that is at least 97% identical to SEQ ID NO: 66.
Embodiment 27. A composition, comprising: (a) an isolated and purified Veillonella atypica having deposit accession number PTA-126860, or a strain having all of the identifying characteristics of Veillonella atypica PTA-126860, or a mutant thereof,
(b) an isolated and purified Lactobacillus plantarum comprising a 16S rRNA nucleic acid sequence that is at least 97% identical to SEQ ID NO: 61;
(c) an isolated and purified Lactobacillus acidophilus comprising a 16S rRNA nucleic acid sequence that is at least 97% identical to SEQ ID NO: 62;
(d) an isolated and purified Lactobacillus rhamnosus comprising a 16S rRNA nucleic acid sequence that is at least 97% identical to SEQ ID NO: 63;
(e) an isolated and purified Bifidobacterium longum comprising a 16S rRNA nucleic acid sequence that is at least 97% identical to SEQ ID NO: 64; and
(f) an isolated and purified Bifidobacterium lactis comprising a 16S rRNA nucleic acid sequence that is at least 97% identical to SEQ ID NO: 65.
Embodiment 28. A composition, comprising: (a) an isolated and purified Veillonella atypica having deposit accession number PTA-126860, or a strain having all of the identifying characteristics of Veillonella atypica PTA-126860, or a mutant thereof,
(b) an isolated and purified Lactobacillus plantarum comprising a 16S rRNA nucleic acid sequence that is at least 97% identical to SEQ ID NO: 61;
(c) an isolated and purified Lactobacillus acidophilus comprising a 16S rRNA nucleic acid sequence that is at least 97% identical to SEQ ID NO: 62; and
(d) an isolated and purified Lactobacillus rhamnosus comprising a 16S rRNA nucleic acid sequence that is at least 97% identical to SEQ ID NO: 63.
Embodiment 29. The composition of any one of embodiments 16-28, further comprising a pharmaceutically acceptable carrier.
Embodiment 30. A method of altering the microbiome of a subject, comprising administering an effective dose of the Veillonella dispar of any one of embodiments 1-4 and 13; Veillonella parvula of any one of embodiments 5-8 and 14; Veillonella atypica of any one of embodiments 9-12 and 15; and/or the composition of any one of embodiments 16-29 to the subject.
Embodiment 31. A method of reducing the level of lactic acid and/or lactates in blood of a subject, comprising administering an effective dose of the Veillonella dispar of any one of embodiments 1-4 and 13; Veillonella parvula of any one of embodiments 5-8 and 14; Veillonella atypica of any one of embodiments 9-12 and 15; and/or the composition of any one of embodiments 16-29 to the subject.
Embodiment 32. A method of increasing the level of propionic acid and/or propionates in blood of a subject, comprising administering an effective dose of the Veillonella dispar of any one of embodiments 1-4 and 13; Veillonella parvula of any one of embodiments 5-8 and 14; Veillonella atypica of any one of embodiments 9-12 and 15; and/or the composition of any one of embodiments 16-29 to the subject.
Embodiment 33. A method of increasing the level of acetic acid and/or acetates in blood of a subject, comprising administering an effective dose of the Veillonella dispar of any one of embodiments 1-4 and 13; Veillonella parvula of any one of embodiments 5-8 and 14; Veillonella atypica of any one of embodiments 9-12 and 15; and/or the composition of any one of embodiments 16-29 to the subject.
Embodiment 34. A method of enhancing exercise endurance of a subject, comprising administering an effective dose of the Veillonella dispar of any one of embodiments 1-4 and 13; Veillonella parvula of any one of embodiments 5-8 and 14; Veillonella atypica of any one of embodiments 9-12 and 15; and/or the composition of any one of embodiments 16-29 to the subject.
Embodiment 35. A method of improving the athletic performance of a subject, comprising administering an effective dose of the Veillonella dispar of any one of embodiments 1-4 and 13; Veillonella parvula of any one of embodiments 5-8 and 14; Veillonella atypica of any one of embodiments 9-12 and 15; and/or the composition of any one of embodiments 16-29 to the subject.
Embodiment 36. A method of reducing inflammation in a subject in need thereof, comprising administering an effective dose of the Veillonella dispar of any one of embodiments 1-4 and 13; Veillonella parvula of any one of embodiments 5-8 and 14; Veillonella atypica of any one of embodiments 9-12 and 15; and/or the composition of any one of embodiments 16-29 to the subject.
Embodiment 37. A method of enhancing recovery from physical exercise in a subject in need thereof, comprising administering an effective dose of the Veillonella dispar of any one of embodiments 1-4 and 13; Veillonella parvula of any one of embodiments 5-8 and 14; Veillonella atypica of any one of embodiments 9-12 and 15; and/or the composition of any one of embodiments 16-29 to the subject.
Embodiment 38. A method of increasing muscle mass and/or muscular strength of a subject in need thereof, comprising administering an effective dose of the Veillonella dispar of any one of embodiments 1-4 and 13; Veillonella parvula of any one of embodiments 5-8 and 14; Veillonella atypica of any one of embodiments 9-12 and 15; and/or the composition of any one of embodiments 16-29 to the subject.
Embodiment 39. A method of preventing the loss of muscle mass of a subject in need thereof, comprising administering an effective dose of the Veillonella dispar of any one of embodiments 1-4 and 13; Veillonella parvula of any one of embodiments 5-8 and 14; Veillonella atypica of any one of embodiments 9-12 and 15; and/or the composition of any one of embodiments 16-29 to the subject.
Embodiment 40. The method of any one of embodiments 30-39, wherein the administration is via oral, enteric, gastrointestinal, or rectal route.
Embodiment 41. The method of any one of embodiments 30-40, wherein the subject is a human subject.
Embodiment 42. The method of any one of embodiments 30-41, wherein the dose is in the range of about 10⁴ CFU to about 10¹⁶ CFU.
Embodiment 43. The method of any one of embodiments 30-42, wherein the dose is in the range of about 10⁹ to about 10¹¹ CFU.
Embodiment 44. The method of any one of embodiments 30-43, wherein the dose is in the range of about 5×10⁹ CFU to about 10¹⁰ CFU.

What is claimed is:

1. An isolated and purified Veillonella dispar, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 98.6% sequence identity to SEQ ID NO: 2; (ii) VR2 comprises a nucleic acid sequence with at least 98.6% sequence identity to SEQ ID NO: 3; (iii) VR3 comprises a nucleic acid sequence with at least 98.6% sequence identity to SEQ ID NO: 4; (iv) VR4 comprises a nucleic acid sequence with at least 98.6% sequence identity to SEQ ID NO: 5; (v) VR5 comprises a nucleic acid sequence with at least 98.6% sequence identity to SEQ ID NO: 6; (vi) VR6 comprises a nucleic acid sequence with at least 98.6% sequence identity to SEQ ID NO: 7; (vii) VR7 comprises a nucleic acid sequence with at least 98.6% sequence identity to SEQ ID NO: 8; (viii) VR8 comprises a nucleic acid sequence with at least 98.6% sequence identity to SEQ ID NO: 9; and (ix) VR9 comprises a nucleic acid sequence with at least 98.6% sequence identity to SEQ ID NO: 10.

2. The Veillonella dispar of claim 1, wherein the nucleic acid sequence comprises at least one constant region (CR) selected from the group consisting of: CR1, CR2, CR3, CR4, CR5, CR6, CR7, CR8, CR9, CR10, and any combination thereof, wherein (i) CR1 comprises a nucleic acid sequence with at least 80% sequence identity to SEQ ID NO: 31; (ii) CR2 comprises a nucleic acid sequence with at least 80% sequence identity to SEQ ID NO: 32; (iii) CR3 comprises a nucleic acid sequence with at least 80% sequence identity to SEQ ID NO: 33; (iv) CR4 comprises a nucleic acid sequence with at least 80% sequence identity to SEQ ID NO:34; (v) CR5 comprises a nucleic acid sequence with at least 80% sequence identity to SEQ ID NO: 35; (vi) CR6 comprises a nucleic acid sequence with at least 80% sequence identity to SEQ ID NO: 36; (vii) CR7 comprises a nucleic acid sequence with at least 80% sequence identity to SEQ ID NO: 37; (viii) CR8 comprises a nucleic acid sequence with at least 80% sequence identity to SEQ ID NO: 38; (ix) CR9 comprises a nucleic acid sequence with at least 80% sequence identity to SEQ ID NO: 39; and (x) CR10 comprises a nucleic acid sequence with at least 80% sequence identity to SEQ ID NO: 40.

3. The Veillonella dispar of claim 1, wherein the nucleic acid sequence comprises at least 98.6% sequence identity to SEQ ID NO: 1.

4. An isolated and purified Veillonella dispar, comprising: a 16S rRNA gene comprising a nucleic acid sequence with at least 98.6% sequence identity to SEQ ID NO: 1.

5. An isolated and purified Veillonella parvula, comprising: a 16S rRNA gene comprising a nucleic acid sequence comprising at least one variable region (VR) selected from the group consisting of VR1, VR2, VR3, VR4, VR5, VR6, VR7, VR8, VR9, and any combination thereof, wherein (i) VR1 comprises a nucleic acid sequence with at least 98.6% sequence identity to SEQ ID NO: 12; (ii) VR2 comprises a nucleic acid sequence with at least 98.6% sequence identity to SEQ ID NO: 13; (iii) VR3 comprises a nucleic acid sequence with at least 98.6% sequence identity to SEQ ID NO: 14; (iv) VR4 comprises a nucleic acid sequence with at least 98.6% sequence identity to SEQ ID NO: 15; (v) VR5 comprises a nucleic acid sequence with at least 98.6% sequence identity to SEQ ID NO: 16; (vi) VR6 comprises a nucleic acid sequence with at least 98.6% sequence identity to SEQ ID NO: 17; (vii) VR7 comprises a nucleic acid sequence with at least 98.6% sequence identity to SEQ ID NO: 18; (viii) VR8 comprises a nucleic acid sequence with at least 98.6% sequence identity to SEQ ID NO: 19; and (ix) VR9 comprises a nucleic acid sequence with at least 98.6% sequence identity to SEQ ID NO: 20.

6. The Veillonella parvula of claim 5, wherein the nucleic acid sequence comprises at least one constant region (CR) selected from the group consisting of: CR1, CR2, CR3, CR4, CR5, CR6, CR7, CR8, CR9, CR10, and any combination thereof, wherein (i) CR1 comprises a nucleic acid sequence with at least 80% sequence identity to SEQ ID NO: 41; (ii) CR2 comprises a nucleic acid sequence with at least 80% sequence identity to SEQ ID NO: 42; (iii) CR3 comprises a nucleic acid sequence with at least 80% sequence identity to SEQ ID NO: 43; (iv) CR4 comprises a nucleic acid sequence with at least 80% sequence identity to SEQ ID NO: 44; (v) CR5 comprises a nucleic acid sequence with at least 80% sequence identity to SEQ ID NO: 45; (vi) CR6 comprises a nucleic acid sequence with at least 80% sequence identity to SEQ ID NO: 46; (vii) CR7 comprises a nucleic acid sequence with at least 80% sequence identity to SEQ ID NO: 47; (viii) CR8 comprises a nucleic acid sequence with at least 80% sequence identity to SEQ ID NO: 48; (ix) CR9 comprises a nucleic acid sequence with at least 80% sequence identity to SEQ ID NO: 49; and (x) CR10 comprises a nucleic acid sequence with at least 80% sequence identity to SEQ ID NO: 50.

7. The Veillonella parvula of claim 5, wherein the nucleic acid sequence comprises at least 98.6% sequence identity to SEQ ID NO: 11.

8. An isolated and purified Veillonella parvula, comprising: a 16S rRNA gene comprising a nucleic acid sequence with at least 98.6% sequence identity to SEQ ID NO: 11.

9. An isolated and purified Veillonella atypica, comprising: a 16S rRNA gene comprising a nucleic acid sequence comprising at least one variable region (VR) selected from the group consisting of: VR1, VR2, VR3, VR4, VR5, VR6, VR7, VR8, VR9, and any combination thereof, wherein (i) VR1 comprises a nucleic acid sequence with at least 98.6% sequence identity to SEQ ID NO: 22; (ii) VR2 comprises a nucleic acid sequence with at least 98.6% sequence identity to SEQ ID NO: 23; (iii) VR3 comprises a nucleic acid sequence with at least 98.6% sequence identity to SEQ ID NO: 24; (iv) VR4 comprises a nucleic acid sequence with at least 98.6% sequence identity to SEQ ID NO: 25; (v) VR5 comprises a nucleic acid sequence with at least 98.6% sequence identity to SEQ ID NO: 26; (vi) VR6 comprises a nucleic acid sequence with at least 98.6% sequence identity to SEQ ID NO: 27; (vii) VR7 comprises a nucleic acid sequence with at least 98.6% sequence identity to SEQ ID NO: 28; (viii) VR8 comprises a nucleic acid sequence with at least 98.6% sequence identity to SEQ ID NO: 29; and (ix) VR9 comprises a nucleic acid sequence with at least 98.6% sequence identity to SEQ ID NO: 30.

10. The Veillonella atypica of claim 9, wherein the nucleic acid sequence comprises at least one constant region (CR) selected from the group consisting of CR1, CR2, CR3, CR4, CR5, CR6, CR7, CR8, CR9, CR10 and any combination thereof, wherein (i) CR1 comprises a nucleic acid sequence with at least 80% sequence identity to SEQ ID NO: 51; (ii) CR2 comprises a nucleic acid sequence with at least 80% sequence identity to SEQ ID NO: 52; (iii) CR3 comprises a nucleic acid sequence with at least 80% sequence identity to SEQ ID NO: 53; (iv) CR4 comprises a nucleic acid sequence with at least 80% sequence identity to SEQ ID NO: 54; (v) CR5 comprises a nucleic acid sequence with at least 80% sequence identity to SEQ ID NO: 55; (vi) CR6 comprises a nucleic acid sequence with at least 80% sequence identity to SEQ ID NO: 56; (vii) CR7 comprises a nucleic acid sequence with at least 80% sequence identity to SEQ ID NO: 57; (viii) CR8 comprises a nucleic acid sequence with at least 80% sequence identity to SEQ ID NO: 58; (ix) CR9 comprises a nucleic acid sequence with at least 80% sequence identity to SEQ ID NO: 59; and (x) CR10 comprises a nucleic acid sequence with at least 80% sequence identity to SEQ ID NO: 60.

11. The Veillonella atypica of claim 9, wherein the nucleic acid sequence comprises at least 98.6% sequence identity to SEQ ID NO: 21.

12. An isolated and purified Veillonella atypica, comprising: a 16S rRNA gene comprising a nucleic acid sequence with at least 98.6% sequence identity to SEQ ID NO: 21.

13. An isolated and purified Veillonella dispar having deposit accession number PTA-126861, or a strain having all of the identifying characteristics of Veillonella dispar PTA-126861, or a mutant thereof.

14. An isolated and purified Veillonella parvula having deposit accession number PTA-126859, or a strain having all of the identifying characteristics of Veillonella parvula PTA-126859, or a mutant thereof.

15. An isolated and purified Veillonella atypica having deposit accession number PTA-126860, or a strain having all of the identifying characteristics of Veillonella atypica PTA-126860, or a mutant thereof.

16. A composition comprising the Veillonella dispar of claim 1.

17. A composition comprising the Veillonella parvula of claim 5.

18. A composition comprising the Veillonella atypica of claim 9.

19. A composition comprising the Veillonella sp. strain of any one of claims 1-15.

20. The composition of claim 16, 17 or 18, wherein the composition comprises one or more lactate producing bacteria.

21. The compositions of claim 20, wherein the lactate-producing bacteria belongs to the genus Lactobacillus or Bifidobacterium.

22. The composition of claim 21, wherein the lactate-producing bacteria is Lactobacillus plantarum, Lactobacillus acidophilus, Lactobacillus rhamnosus, Bifidobacterium longum, Bifidobacterium lactis, or any combination thereof.

23. The composition of claim 20, wherein the composition produces more acetate than the Veillonella sp. strain.

24. A composition, comprising: a Veillonella sp. strain of any one of claims 1-15, and any one or more of the following strains:
(a) an isolated and purified Lactobacillus plantarum comprising a 16S rRNA nucleic acid sequence that is at least 97% identical to SEQ ID NO: 61;
(b) an isolated and purified Lactobacillus acidophilus comprising a 16S rRNA nucleic acid sequence that is at least 97% identical to SEQ ID NO: 62;
(c) an isolated and purified Lactobacillus rhamnosus comprising a 16S rRNA nucleic acid sequence that is at least 97% identical to SEQ ID NO: 63;
(d) an isolated and purified Bifidobacterium longum comprising a 16S rRNA nucleic acid sequence that is at least 97% identical to SEQ ID NO: 64;
(e) an isolated and purified Bifidobacterium lactis comprising a 16S rRNA nucleic acid sequence that is at least 97% identical to SEQ ID NO: 65; and
(f) an isolated and purified Lactobacillus paracasei comprising a 16S rRNA nucleic acid sequence that is at least 97% identical to SEQ ID NO: 66.

25. A composition, comprising: a Veillonella sp. strain of any one of claims 1-15, and any one or more of the following strains:
(a) an isolated and purified Lactobacillus plantarum comprising a 16S rRNA nucleic acid sequence that is at least 97% identical to SEQ ID NO: 61;
(b) an isolated and purified Lactobacillus acidophilus comprising a 16S rRNA nucleic acid sequence that is at least 97% identical to SEQ ID NO: 62;
(c) an isolated and purified Lactobacillus rhamnosus comprising a 16S rRNA nucleic acid sequence that is at least 97% identical to SEQ ID NO: 63; and
(f) an isolated and purified Lactobacillus paracasei comprising a 16S rRNA nucleic acid sequence that is at least 97% identical to SEQ ID NO: 66.

26. A composition, comprising: (a) an isolated and purified Veillonella atypica having deposit accession number PTA-126860, or a strain having all of the identifying characteristics of Veillonella atypica PTA-126860, or a mutant thereof,
(b) an isolated and purified Lactobacillus plantarum comprising a 16S rRNA nucleic acid sequence that is at least 97% identical to SEQ ID NO: 61;
(c) an isolated and purified Lactobacillus acidophilus comprising a 16S rRNA nucleic acid sequence that is at least 97% identical to SEQ ID NO: 62;
(d) an isolated and purified Lactobacillus rhamnosus comprising a 16S rRNA nucleic acid sequence that is at least 97% identical to SEQ ID NO: 63;
(e) an isolated and purified Bifidobacterium longum comprising a 16S rRNA nucleic acid sequence that is at least 97% identical to SEQ ID NO: 64; and
(f) an isolated and purified Bifidobacterium lactis comprising a 16S rRNA nucleic acid sequence that is at least 97% identical to SEQ ID NO: 65.

27. A composition, comprising: (a) an isolated and purified Veillonella atypica having deposit accession number PTA-126860, or a strain having all of the identifying characteristics of Veillonella atypica PTA-126860, or a mutant thereof,
(b) an isolated and purified Lactobacillus plantarum comprising a 16S rRNA nucleic acid sequence that is at least 97% identical to SEQ ID NO: 61;
(c) an isolated and purified Lactobacillus acidophilus comprising a 16S rRNA nucleic acid sequence that is at least 97% identical to SEQ ID NO: 62; and
(d) an isolated and purified Lactobacillus rhamnosus comprising a 16S rRNA nucleic acid sequence that is at least 97% identical to SEQ ID NO: 63.

28. The composition of claim 16, 17 or 18, wherein the composition is a food composition, a beverage composition or a dietary supplement composition.

29. The composition of claim 16, 17 or 18, comprising a pharmaceutically acceptable carrier.

30. A method of altering the microbiome of a subject, comprising administering an effective dose of the Veillonella dispar of any one of claims 1-4 and 13; Veillonella parvula of any one of claims 5-8 and 14; Veillonella atypica of any one of claims 9-12 and 15; and/or the composition of claim 16, 17 or 18 to the subject.

31. A method of reducing the level of lactic acid and/or lactates in blood of a subject, comprising administering an effective dose of the Veillonella dispar of any one of claims 1-4 and 13; Veillonella parvula of any one of claims 5-8 and 14; Veillonella atypica of any one of claims 9-12 and 15; and/or the composition of claim 16, 17 or 18 to the subject.

32. A method of increasing the level of propionic acid and/or propionates in blood of a subject, comprising administering an effective dose of the Veillonella dispar of any one of claims 1-4 and 13; Veillonella parvula of any one of claims 5-8 and 14; Veillonella atypica of any one of claims 9-12 and 15; and/or the composition of claim 16, 17 or 18 to the subject.

33. A method of increasing the level of acetic acid and/or acetates in blood of a subject, comprising administering an effective dose of the Veillonella dispar of any one of claims 1-4 and 13; Veillonella parvula of any one of claims 5-8 and 14; Veillonella atypica of any one of claims 9-12 and 15; and/or the composition of claim 16, 17 or 18 to the subject.

34. A method of enhancing exercise endurance of a subject, comprising administering an effective dose of the Veillonella dispar of any one of claims 1-4 and 13; Veillonella parvula of any one of claims 5-8 and 14; Veillonella atypica of any one of claims 9-12 and 15; and/or the composition of claim 16, 17 or 18 to the subject.

35. A method of improving the athletic performance of a subject, comprising administering an effective dose of the Veillonella dispar of any one of claims 1-4 and 13; Veillonella parvula of any one of claims 5-8 and 14; Veillonella atypica of any one of claims 9-12 and 15; and/or the composition of claim 16, 17 or 18 to the subject.

36. A method of reducing inflammation in a subject in need thereof, comprising administering an effective dose of the Veillonella dispar of any one of claims 1-4 and 13; Veillonella parvula of any one of claims 5-8 and 14; Veillonella atypica of any one of claims 9-12 and 15; and/or the composition of claim 16, 17 or 18 to the subject.

37. A method of enhancing recovery from physical exercise in a subject in need thereof, comprising administering an effective dose of the Veillonella dispar of any one of claims 1-4 and 13; Veillonella parvula of any one of claims 5-8 and 14; Veillonella atypica of any one of claims 9-12 and 15; and/or the composition of claim 16, 17 or 18 to the subject.

38. A method of increasing muscle mass and/or muscular strength of a subject in need thereof, comprising administering an effective dose of the Veillonella dispar of any one of claims 1-4 and 13; Veillonella parvula of any one of claims 5-8 and 14; Veillonella atypica of any one of claims 9-12 and 15; and/or the composition of claim 16, 17 or 18 to the subject.

39. A method of preventing the loss of muscle mass of a subject in need thereof, comprising administering an effective dose of the Veillonella dispar of any one of claims 1-4 and 13; Veillonella parvula of any one of claims 5-8 and 14; Veillonella atypica of any one of claims 9-12 and 15; and/or the composition of claim 16, 17 or 18 to the subject.

40. The method of claim 30, wherein the administration is via oral, enteric, gastrointestinal, or rectal route.

41. The method of claim 30, wherein the subject is a human subject.

42. The method of claim 30, wherein the dose is in the range of about 10⁴ CFU to about 10¹⁶ CFU.

43. The method of claim 30, wherein the dose is in the range of about 10⁹ to about 10¹¹ CFU.

44. The method of claim 30, wherein the dose is in the range of about 5×10⁹ CFU to about 10¹⁰ CFU.
